(19) United States
(12) Patent Application Publication
(10) Pub. No.: US 2010/0137202 A1
(43) Pub. Date: Jun. 3, 2010

(54) COMPOSITIONS FOR REPROGRAMMING A CELL AND USES THEREFOR

(75) Inventor: Li-Jun Yang, Gainesville, FL (US)

Correspondence Address: EDWARDS ANGELL PALMER & DODGE LLP, P.O. BOX 55874, BOSTON, MA, 02205, US

(73) Assignee: University of Florida Research Foundation, Gainesville, FL (US)

(21) Appl. No.: 12/374,018

(22) PCT Filed: Jul. 19, 2007
(86) PCT No.: PCT/US2007/016402

§ 371 Date: Feb. 5, 2010

Related U.S. Application Data
(60) Provisional application No. 60/832,070, filed on Jul. 19, 2006.

(51) Int. Cl. A61K 38/16; C12N 5/071; A61P 3/00

(52) U.S. Cl. 514/12; 435/375

(57) ABSTRACT

The present invention generally provides therapeutic compositions and methods for treating a disease, disorder, or injury characterized by a deficiency in the number or biological activity of a cell of interest. The method provides compositions for generating reprogrammed cells or for increasing regeneration in a cell, tissue, or organ of interest. Such methods are useful for treating subjects having a deficiency in a particular cell type or in a polypeptide produced by that cell type. In particular, the invention provides prophylactic and therapeutic methods and compositions for ameliorating or preventing hyperglycemia associated with type I and type II diabetes and related complications.

### CROSS-REFERENCE TO RELATED APPLICATION

This application claims the benefit of U.S. Provisional Application No. 60/832,070, filed on Jul. 19, 2006, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: DK064054 and DK071831. The government may have certain rights in the invention.

### BACKGROUND OF THE INVENTION

Because current methods for treating type 1 diabetes (T1D) are ineffective in preventing long-term complications, investigators are seeking alternative therapies, including the use of insulin-producing cells (IPCs) to restore euglycemia. Although T1D can be cured by transplantation of functional β-cells, islet transplantation is hampered by the scarcity of donor islets and a need for life-long immunosuppressive therapy to reduce risks of graft rejection.

To cure Type 1 diabetes, researchers are actively pursuing reliable strategies for achieving the directed differentiation of stem cells into insulin-producing surrogates for pancreatic beta-cells (IPCs). Genetic modulation with recombinant DNA technology is a highly promising approach to direct stem-cell differentiation into a desired lineage, particularly with respect to liver-to-endocrine-pancreas transdifferentiation. This approach has been used to generate IPCs from liver and liver stem cells, in both in vitro and in vivo experiments. Although these studies offer great promise in using liver as potential autologous donor cells for cell therapy in treatment of type 1 diabetes, the use of genetic manipulation in human clinical therapy has proved controversial. Accordingly, compositions and methods for producing insulin-producing cells that do not rely on gene therapy are urgently required.

### SUMMARY OF THE INVENTION

As described below, the present invention features therapies for the treatment or prevention of diseases, disorders or injuries (e.g., diabetes), characterized by a deficiency in the number or biological activity of a cell of interest (e.g., a pancreatic cell).

In one aspect, the invention generally provides a method for reprogramming a cell (e.g., an adult cell or embryonic stem cell), the method involving contacting the adult cell with a transcription factor polypeptide or fragment thereof fused to or comprising a protein transduction domain; and altering the expression level of at least one polypeptide (e.g., a pancreatic transcription factor polypeptide) in the adult cell, thereby reprogramming the cell.

In another aspect, the invention provides a method for generating an insulin producing cell, the method involving contacting a cell (e.g., an adult cell or embryonic stem cell), with a pancreatic transcription factor or fragment thereof fused to a protein transduction domain; and increasing the expression of insulin in the cell, thereby generating an insulin producing cell.

In yet another aspect, the invention provides a method for inducing the regeneration of an insulin producing cell, the method comprising contacting a pancreatic cell with a fusion protein comprising a Pdx-1 polypeptide, a VP16 activation domain, and a protein transduction domain, or biologically active fragments thereof, thereby inducing the pancreatic cell to regenerate.

In yet another aspect, the invention provides a method for generating an insulin producing cell, the method comprising contacting a liver-derived cell with a fusion protein comprising a Pdx-1 polypeptide, a VP16 activation domain, and a protein transduction domain, or biologically active fragments thereof, thereby generating an insulin producing cell. In one embodiment, the liver-derived cell is contacted in vitro or in vivo. In another embodiment, the fusion protein is provided via the portal vein, or via a branch thereof, such that the fusion protein is directed to a particular lobe of the liver. In yet another embodiment, the insulin-producing cell is generated by the transdifferentiation of the liver-derived cell.

In yet another aspect, the invention provides a method of ameliorating hyperglycemia in a subject in need thereof, the method involving contacting an adult cell of the subject with a pancreatic transcription factor or fragment thereof fused to a protein transduction domain; and increasing the expression of insulin in the adult cell, thereby generating an insulin producing cell.

In yet another aspect, the invention provides a method of ameliorating hyperglycemia in a subject in need thereof, the method involving contacting an adult pancreatic cell of the subject with a fusion protein comprising a Pdx-1 polypeptide, a VP16 activation domain, and a protein transduction domain, or biologically active fragments thereof; and inducing the adult pancreatic cell to regenerate, thereby ameliorating hyperglycemia in the subject.

In still another aspect, the invention provides a method of ameliorating hyperglycemia in a subject (e.g., a human or animal patient having type 1 or type 2 diabetes) in need thereof, the method comprising contacting a liver-derived cell with a fusion protein comprising a Pdx-1 polypeptide, a VP16 activation domain, and a protein transduction domain, or biologically active fragments thereof; and generating an insulin producing cell, thereby ameliorating hyperglycemia in the subject. In one embodiment, the liver-derived cell is a hepatocyte or liver-derived stem cell. In yet another embodiment, the cell fails to express a detectable level of insulin prior to contact with the fusion protein. In yet another embodiment, the method reduces blood glucose level in the subject. In yet another embodiment, the method normalizes blood glucose level in the subject. In still other embodiments, the method further comprises administering an effective amount of a fusion protein comprising an Ngn3 polypeptide, a protein transduction domain, or biologically active fragments thereof.

In another aspect, the invention provides a fusion polypeptide containing or consisting essentially of a polypeptide having at least 85%, 90%, 95%, or even 100% amino acid identity to an early or late pancreatic transcription factor (e.g., Pdx-1, Ngn3, Pax4, NeuroD1, Nkx2.2, Nkx6.1, Is11, and Pax6); and a protein transduction domain (e.g., HIV-1 TAT PTD domain, a polyarginine sequence, a VP22 domain, or an antennapedia protein transduction domain), analog, or fragment thereof, wherein expression of the fusion polypeptide in a cell alters the expression of at least one polypeptide relative to a corresponding control cell. In one embodiment, the fusion polypeptide further comprises a Herpes simplex virus VP16 activation domain, or fragment or analog thereof, a sequence tag for purification (e.g., a hexahistidine tag), or an antigenic domain that specifically binds an antibody (e.g., a V5 domain). In yet another embodiment, the fusion polypeptide comprises or consists essentially of a human Pdx-1 polypeptide or fragment thereof; a Herpes simplex virus VP16 activation domain; and a protein transduction domain, or biologically active fragments thereof.

In yet another aspect, the fusion polypeptide contains a pancreatic transcription factor promoter and a detectable domain. In various embodiments, the promoter is selected from any one or more of Pdx-1, Ngn3, Pax4, NeuroD1, Nkx2.2, Nkx6.1, Is11, and Pax6; in other embodiments; the detectable domain is selected from the any one or more of green fluorescent protein, red fluorescent protein, glucuronidase (GUS), luciferase, chloramphenicol transacetylase (CAT), and beta-galactosidase. In other embodiments, the polypeptide further comprises an amino acid sequence tag that facilitates purification of the polypeptide, such as a hexahistidine tag or GST.

In other aspects, the invention provides expression vectors comprising a nucleic acid sequence encoding a polypeptide of any previous aspect. In one embodiment, the vector contains a promoter operably linked to the nucleic acid sequence. In one embodiment, the promoter is positioned for expression in a bacterial or mammalian cell.

In yet another aspect, the invention provides a host cell comprising the expression vector of any previous aspect. In one embodiment, the cell is a prokaryotic or eukaryotic cell. In other embodiments, the cell is selected from any one or more of a bacterial cell, a mammalian cell, an insect cell, and a yeast cell.

In yet another aspect, host cell (e.g., mammalian, human) contains the fusion polypeptide of any previous aspect. In still other embodiments, the cell is selected from any one or more of adipocytes, bone marrow derived cells, epidermal cells, endothelial cells, fibroblasts, hematopoietic cells, hepatocytes, myocytes, neurons, pancreatic cells, and their progenitor cells or stem cells. In other embodiments, the host cell further comprises a second fusion polypeptide, such as a fusion polypeptide that contains Ngn3 and a protein transduction domain, or biologically active fragments thereof.

In yet another aspect, the invention provides a tissue containing the host cell of any previous aspect.

In yet another aspect, the invention provides an organ containing the host cell of any previous aspect.

In yet another aspect, the invention provides a matrix comprising a host cell of any previous aspect.

In yet another aspect, the invention provides a method for producing a recombinant polypeptide of any previous aspect, the method comprising providing a cell transformed with the isolated nucleic acid molecule of any previous aspect positioned for expression in the cell; culturing the cell under conditions for expressing the nucleic acid molecule; and isolating the polypeptide.

In yet another aspect, the invention provides a pharmaceutical composition comprising an effective amount of a polypeptide of any previous aspect or a host cell of a previous aspect in a pharmaceutically acceptable excipient. In other embodiments, the composition further contains an effective amount of a fusion polypeptide comprising an Ngn3 amino acid sequence and a protein transduction domain, or biologically active fragments thereof.

In yet another aspect, the invention provides a packaged pharmaceutical containing a polypeptide of any previous aspect or a host cell of any previous aspect; and instructions for using the polypeptide or cell to ameliorate hyperglycemia in a subject (e.g., hyperglycemia related to type 1 or type 2 diabetes).

In yet another aspect, the invention provides a kit for treating hyperglycemia (e.g., hyperglycemia related to type 1 or type 2 diabetes), the kit comprising an effective amount of a fusion polypeptide of any previous aspect or a host cell of any previous aspect, and instructions for use thereof.

In yet another aspect, the invention provides an adeno-associated viral (AAV) vector comprising a pancreatic transcription factor that is any one or more of Pdx-1, Ngn3, Pax4, NeuroD1, Nkx2.2, Nkx6.1, Is11, Pax6, and MafA, wherein the polypeptide is operably linked to a promoter positioned for expression in a mammalian cell.

In yet another aspect, the invention provides a method of ameliorating hyperglycemia in a subject in need thereof, the method involving contacting a cell of the subject with the AAV vector a previous aspect; and inducing the cell to express insulin, thereby ameliorating hyperglycemia in the subject.

In yet another aspect, the invention provides a method of ameliorating hyperglycemia in a subject in need thereof, the method involving contacting a liver-derived cell of the subject with the AAV vector of the previous aspect; and inducing the cell to express insulin, thereby ameliorating hyperglycemia in the subject.

In yet another aspect, the invention provides a method of ameliorating hyperglycemia in a subject in need thereof, the method involving contacting a cell of the subject with the AAV vector a previous aspect and inducing the cell to express insulin, thereby ameliorating hyperglycemia in the subject.

In yet another aspect, the invention provides a method of ameliorating hyperglycemia in a subject in need thereof, the method involving contacting a liver-derived cell of the subject with the AAV vector of a previous aspect; and inducing the cell to express insulin, thereby ameliorating hyperglycemia in the subject.

In yet another aspect, the invention provides a host cell (e.g., an adult cell, embryonic stem cell, liver or pancreatic cell) containing the AAV vector or polypeptide (e.g., fusion polypeptide) of a previous aspect. In one embodiment, the cell is one or more of adipocytes, bone marrow derived cells, epidermal cells, endothelial cells, fibroblasts, hematopoietic cells, hepatocytes, myocytes, neurons, pancreatic cells, and their progenitor cells or stem cells.

In yet another aspect, the invention provides a pharmaceutical composition comprising an effective amount of an adenoviral vector in a pharmaceutically acceptable excipient.

In yet another aspect, the invention provides a method of treating diabetes in a subject, the method comprising contacting the subject with a Pdx1 polypeptide in an amount effective to induce expression of a gene that is any one or more of Pdx1, INGAP, Reg3d, Reg3g, and pancreatitis-associated protein—Pap in a tissue of the subject, thereby treating the diabetes. In one embodiment, the increase in expression is any one or more of: Pdx1 is increased by about 3-4 times, INGAP expression is increased by about 14-15 times, Reg3d expression is increased by about 7-8 times, Reg3g is increased by about 6-7 times, and pancreatitis-associated protein—Pap is increased by 30-35 times. In yet another embodiment, the method increases expression of any one or more of Pdx1, insulin I, glucagon, elastase, IAPP, insulin II, somatostatin, NeuroD, Isl-1 and pancreatic exocrine genes p48 and amylase. In yet another embodiment, the expression of at least two, three, four, or five genes is increased. In yet another embodiment, the expression of all of the genes is increased.

In yet another aspect, the invention provides a method of inducing islet β-cell regeneration in a subject in need thereof, the method comprising administering an effective amount of Pdx1 protein to the subject and inducing the regeneration of an islet β cell. In one embodiment, at least about 1-5 mg/kg body weight PDX1 is administered. In another embodiment, PDX1 is administered via the intravenous or peritoneal system. In another embodiment, the method increases insulin level by at least about 1 to 20 times. In another embodiment, the PDX1 administration increases the expression of any one or more of Pdx1, INGAP, Reg3d, Reg3g, pancreatitis-associated protein—Pap, insulin I, glucagon, elastase, IAPP, insulin II, somatostatin, NeuroD, Isl-1 and pancreatic exocrine genes p48 and amylase.

In still other aspects, the invention provides host cells (e.g., adult cell, embryonic stem cells, liver or pancreatic cells) comprising the AAV vectors as well as pharmaceutical compositions comprising an effective amount of such vectors or cells in a pharmaceutically acceptable excipient.

In various embodiments of any previous aspect, the cell is an adult cell (e.g., hepatocyte or liver-derived stem cell) that is any one or more of adipocytes, bone marrow derived cells, epidermal cells, endothelial cells, fibroblasts, hematopoietic cells, hepatocytes, myocytes, neurons, pancreatic cells, and their progenitor cells or stem cells. In yet other embodiments, the cell is contacted in vitro or in vivo. In still other embodiments of any previous aspect, the alteration is an increase (e.g., 5%, 10%, 25%, 50%, 75%, 85%, 95% or more) in the level of a polypeptide that is not detectably expressed in a corresponding control cell. In still other embodiments, the reprogrammed cell expresses insulin. In various embodiments of any previous aspect, the transcription factor is any one or more of Pdx-1, Pdx-1/VP16, Ngn3, Pax4, NeuroD1, Nkx2.2, Nkx6.1, Is11, Pax6, and MafA. In still other embodiments, the cell is an embryonic or adult pancreatic cell that is contacted in vitro or in vivo. In still other embodiments of any of the above aspects, the contact increases the number of insulin producing cells. In other embodiments, the contact increases regeneration by replication or neogenesis. In still other embodiments, the method further comprises contacting the cell with a fusion protein comprising Ngn3 and a protein transduction domain or biologically active fragments thereof. In still other embodiments, the method further comprises the step of obtaining the polypeptide. In still other embodiments of any previous aspect, the administration method provides for the sequential administration of one or more pancreatic transcription factors, including the administration of any one or more of an effective amount of Pdx1 protein in a cell for at least about 0-4 (e.g., 0, 1, 2, 3, or 4) days; administering next Ngn3, such that an effective amount of Ngn3 is present for at least about 2-6 (e.g., 2, 3, 4, 5, or 6) days; administering next Pax4, such that an effective amount of Pax4 is present for at least about 4-8 (e.g., 4, 5, 6, 7, 8) days; administering persistently Pdx1, such that an effective amount of Pdx1 is sustained in the cell, wherein the administration of Pdx1 occurs while Pax4 is present in the cell.

In yet other embodiments of any previous aspect, the fusion protein contains or consists essentially of a pancreatic transcription factor (e.g., human or mouse) that is an early factor or a later factor (e.g., Pdx-1, Ngn3, Pax4, NeuroD1, Nkx2.2, Nkx6.1, Is11, Pax6, MafA). In still other embodiments, the fusion polypeptide of any previous aspect is at least 85%, 90%, or 95% identical to a reference sequence, where the sequence comparison is to the overall length of the polypeptide or peptide fragment.

In particular, the invention provides protein therapy for type 1 and type 2 diabetes. Other features and advantages of the invention will be apparent from the detailed description, and from the claims.

### DEFINITIONS

By “Pancreatic and Duodenal Homeobox-1 (Pdx-1) polypeptide” is meant a protein or fragment thereof having at least 85% homology to the sequence provided at GenBank Accession No. NP_{—}032840, AAI11593, or a sequence encoded by NM008814, and having DNA binding or transcriptional regulation activity.

By “Pancreatic and Duodenal Homeobox-1 (Pdx-1)” nucleic acid sequence is meant a nucleic acid sequence encoding PDX-1. Exemplary pdx-1 nucleic acid sequences include BC111592 and NM_{—}008814.

By “NeuroD1 polypeptide” is meant a neurogenic differentiation 1 protein or fragment thereof having at least 85% homology to the sequence provided at GenBank Accession No. NP_{—}002491 or NP_{—}035024 and having DNA binding or transcriptional regulation activity.

By “NeuroD1 nucleic acid molecule” is meant a polynucleotide or fragment thereof that encodes a NeuroD1 polypeptide. Exemplary NeuroD1 nucleic acid molecules include NM_{—}002500 and NM_{—}010894.

By “Neurogenin 3” polypeptide” is meant a protein or fragment thereof having at least 85% homology to the sequence provided at GenBank Accession No. AAK15022 or AAI04328 and having DNA binding or transcriptional regulation activity.

By “Neurogenin 3 nucleic acid molecule” is meant a polynucleotide or fragment thereof that encodes a Neurogenin 3 polypeptide or fragment thereof. Exemplary Neurogenin 3 nucleic acid molecules include AF234829 and BC104327.

By “Pax4 polypeptide” is meant a protein or fragment thereof having at least 85% homology to the sequence provided at GenBank Accession No. AAI07151 or NP_{—}035168 and having DNA binding or transcriptional regulation activity

By “Pax4 nucleic acid molecule” is meant a polynucleotide or fragment thereof that encodes a Pax4 polypeptide. Exemplary Pax4 polypeptides include NM_{—}011038 and BC107150.

By “adult cell” is meant a somatic cell not derived from an embryo or germ cell.

By “inducing regeneration” is meant inducing the generation of a cell, tissue or organ. Methods of regeneration include, but are not limited to, neogenesis, replication, cell proliferation, transdifferentiation, or any other method that involves the production of additional cells that resemble a cell of interest.

By “protein transduction domain” is meant an amino acid sequence that facilitates protein entry into a cell or cell organelle. Exemplary protein transduction domains include but are not limited to a minimal unidecapeptide protein transduction domain (corresponding to residues 47-57 of HIV-1 TAT comprising YGRKKRRQRRR), a polyarginine sequence comprising a number of arginines sufficient to direct entry into a cell (e.g., 3, 4, 5, 6, 7, 8 or 9 arginines), a VP22 domain (Zender et al., Cancer Gene Ther. 2002 June;9(6):489-96), and an antennapedia protein transduction domain (Noguchi et al., Diabetes 2003; 52(7):1732-1737). See, also, Nat Biotechnol. 2001 December;19(12):1173-6.

By “reprogramming” is meant altering a cell such that at least one protein product is produced in the reprogrammed cell that is not produced in the cell prior to reprogramming (or in a corresponding control cell). Typically, the reprogrammed cell has an altered transcriptional or translational profile, such that the reprogrammed cell expresses a set of proteins not expressed in the cell prior to reprogramming (or in a corresponding control cell).

By “liver-derived cell” is meant any cell derived from the liver. Such cells include hepatocytes, liver stem cells, primary or immortalized cultures of liver cells, or any other cell obtained from the liver.

By “pancreatic transcription factor” is meant any transcription factor expressed in a pancreatic tissue. Exemplary pancreatic transcription factors include, but are not limited to, Pdx-1, Pdx-1/VP16, Ngn3, Pax4, NeuroD1, Nkx2.2 (mouse NM_{—}010919, NP_{—}035049; human C075092, AAH75092), Nkx6.1 (mouse NP_{—}659204, AF357883; human P78426, NM_{—}006168) Is11 (mouse NM_{—}021459, NP_{—}067434; human NM_{—}002202, NP_{—}002193), Pax6 (mouse BC036957, AAH36957; human NP_{—}000271, BC011953), and MafA (v-maf musculoaponeurotic fibrosarcoma oncogene homolog A), human NP_{—}963883, NM_{—}201589; mouse NP_{—}919331, NM_{—}194350).

By “transdifferentiation” is meant an alteration in a cell such that the transdifferentiated cell expresses at least one protein of interest not typically expressed in the cell. For example, a liver cell transdifferentiated to an insulin producing cell phenotype expresses insulin or glucagon.

By “VP16 activation domain” is meant an amino acid sequence derived from Herpes simplex virus that increases transcription when appended to a sequence of interest. Exemplary VP16 activation domains are described, for example, by Sadowski, I., Ma, J., Triezenberg, S. and Ptashne, M. (1988). GAL4-VP16 is an unusually potent transcriptional activator. Nature 335, 563-564; and by Triezenberg, S. J., Kingsbury, R. C. and McKnight, S. L. (1988). Functional dissection of VP16, the trans-activator of herpes simplex virus immediate early gene expression. Genes Dev. 2, 718-729. One exemplary VP16 activation domain is highlighted in FIG. 18G.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and most preferably a 50% or greater change in expression levels.

By “analog” is meant a structurally related polypeptide or nucleic acid molecule having the function of a reference polypeptide or nucleic acid molecule.

By “compound” is meant any small molecule chemical compound, antibody, nucleic acid molecule, or polypeptide, or fragments thereof.

In this disclosure, “comprises,” “comprising,” “containing” and “having” and the like can have the meaning ascribed to them in U.S. Patent law and can mean “ includes,” “including,” and the like; “consisting essentially of or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

A “labeled nucleic acid or polypeptide” is one that is bound, either covalently, through a linker or a chemical bond, or noncovalently, through ionic bonds, van der Waals forces, electrostatic attractions, hydrophobic interactions, or hydrogen bonds, to a label such that the presence of the nucleic acid or probe may be detected by detecting the presence of the label bound to the nucleic acid or probe.

By “an effective amount” is meant the amount of an agent required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “fusion protein” is meant a protein that combines at least two amino acid sequence that are not naturally contiguous.

By “identity” is meant the amino acid or nucleic acid sequence identity between a sequence of interest and a reference sequence. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “hybridize” is meant pair to form a double-stranded molecule between complementary polynucleotide sequences (e.g., genes listed in Tables 1 and 2), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “increases or decreases” is meant a positive or negative alteration. Such alterations are by 5%, 10%, 25%, 50%, 75%, 85%, 90% or even by 100% of a reference value.

By “isolated nucleic acid molecule” is meant a nucleic acid (e.g., a DNA) that is free of the genes, which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. In one embodiment, the preparation is at least 75%, 85%, 90%, 95%, or at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “matrix” is meant a medium that provides for the survival, proliferation, or growth of one or more cells. In one embodiment, a matrix is a cell scaffold comprising a biodegradable medium.

By “naturally occurs” is meant is endogenously expressed in a cell of an organism.

By “obtaining” as in “obtaining the polypeptide” is meant synthesizing, purchasing, or otherwise acquiring the polypeptide.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “polypeptide” is meant any chain of amino acids, regardless of length or post-translational modification.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “promoter” is meant a polynucleotide sufficient to direct transcription. Exemplary promoters include nucleic acid sequences of lengths 100, 250, 300, 400, 500, 750, 900, 1000, 1250, and 1500 nucleotides that are upstream (e.g., immediately upstream) of the translation start site.

By “subject” is meant a mammal, including, but not limited to, a human or non-human mammal, such as a bovine, equine, canine, ovine, or feline.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. By “ameliorate” is meant decrease, suppress, attenuate, diminish, arrest, or stabilize the development or progression of a disease.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reference” is meant a standard or control condition.

### BRIEF DESCRIPTION OF THE DRAWINGS

FIGS. 1A and 1B are schematic diagrams. FIG. 1A shows a simplified model for the role of islet transcription factors in endocrine differentiation in the developing pancreas. The proposed position for each TF is based on its timing of expression and timing of predominant functional role, or both. FIG. 1B shows the structure of protein transduction domain (PTD) fusion proteins.

FIGS. 2A, 2B, and 2C show a PTD-Ngn3-V5 fusion protein by Coomassie blue staining (FIG. 2A), Western blot using an anti-V5 antibody (1:5000) FIG. 2B, and by Coomassie blue staining with or without the PTD domain. Bacterial lysates are present in lanes 1 & 2; Ni—NTA purified PTD-Ngn3-V5 fusion protein is present in lane 3, with their position indicated by arrows.

FIGS. 3A to 3D show an analysis of the PTD-Ngn3 fusion protein. FIG. 3A is a Western blot showing a time course that analyses the cellular transduction of the fusion protein. FIG. 2B includes two micrographs showing cells in culture that have transduced the fusion protein. The cells are shown using phase contrast on the left and fluorescence on the right. The fluorescent image shows the intercellular localization of the PTD-Ngn3 fusion protein. FIG. 3C shows the stability of PTD-Ngn3 in culture medium. FIG. 3D is a Western blot showing that the Ngn3 fusion protein retains its biological function and is able to induce the expression of NeuroD and Pax4 target genes.

FIGS. 4A and 4B are SDS-PAGE gels stained with Coomassie blue that show the purification of soluble and insoluble PTD-Ngn3 fusion proteins.

FIGS. 5A and 5B are SDS-PAGE gels stained with Coomassie blue showing a Pdx1 and a PTD-PDX1 fusion protein and a graph, respectively. FIG. 5B shows that intraperitoneal injection of a soluble PTD-Pdx1 fusion protein restores normoglycemia in diabetic mice.

FIG. 6 is a graph showing the effects of a blood glucose challenge test on diabetic mice receiving intraperitoneal injections of the PTD-Pdx-1 fusion protein, of control diabetic mice that received a PTD-GFP fusion protein, or on normal mice. Intraperitoneal injection of the PTD-Pdx1 fusion protein restored the ability of diabetic mice to respond to a blood glucose challenge (squares/line).

FIGS. 7A and 7B show the effects of intraperitoneal injection of the PTD-Pdx-1 protein on blood glucose levels in mice with a 90% pancreatectomy. FIG. 7A is a graph showing that mice that received the PTD-Pdx1 injection showed a reduction in blood glucose level within days of receiving the injection. This effect eventually dissipated when injections of the fusion protein ceased. FIG. 7B includes two photomicrographs showing that PTD-Pdx1VP16 delivered via intraperitoneal injection into diabetic mice promotes pancreatic beta-cell regeneration. Sections of the pancreases taken from mice receiving PTD-GFP (FIG. 7B-a) or PTD-Pdx1-VP16 (FIG. 7B-b) were double immunostained with anti-pHH3 (phosphor-histone H3 protein, red) and anti-insulin (green) antibodies. The nuclei were highlighted in blue by Dapi staining. FIGS. 7B-a and 7B-b shows that many positively-staining mitotic cells are present within islets (arrow) and outside islets of the pancreas in the mouse receiving PTD-Pdx1-VP16 or Pdx1 injections. In contrast, no mitotic (staining) cells were noted in an islet (FIG. 7B-a, arrow) and rare cells in outside islets in the mouse receiving PTD-GFP injections.

FIG. 8 is a graph showing that intraperitoneal injection of a PTD-Pdx1 VP16 fusion protein ameliorated hyperglycemia in Stz-induced diabetic mouse.

FIG. 9 is a graph showing that intraperitoneal injections of both the PTD-Pdx1 VP16 fusion protein and the PTD-Ngn3 fusion proteins reversed hyperglycemia in mice with Streptozotocin-induced diabetes.

FIGS. 10A and 10B are photomicrographs showing that intraperitoneal injections of the PTD-Pdx1-VP16 protein promoted robust pancreatic beta cells regeneration. FIG. 10A shows insulin-positive islet beta cells highlighted by insulin immunostaining in a complete pancreas section. FIG. 10B shows a high-power view of insulin-immunostaining in islets of various sizes (small to large). Some show only single insulin+cells.

FIGS. 11A-11D are photomicrographs. FIG. 11A shows insulin staining in a pancreatic islet beta cell. FIG. 11B shows that insulin positive cells are present in the liver of a mouse that received intraperitoneal injection of a PTD-Pdx1-VP16 fusion protein. FIG. 11C is a section showing a secondary antibody control, which indicates that the staining is specific. FIG. 11D shows that no insulin staining is present in liver section from a mouse that received intraperitoneal injections of a PTD-GFP control fusion protein.

FIG. 12 shows four photomicrographs of insulin-producing cells (IPCs) in liver sections of a mouse that received intraperitoneal injections of a PTD-Pdx1-VP16 and PTD-Ngn3 at day 27 post-injection. Various liver cells producing insulin are present in the liver section (overall 1-2%).

FIGS. 13A and 13B show the effects of AAV-pancreatic transcription factors on blood glucose levels in Stz-induced diabetic mice. FIG. 13A shows GFP protein expression in the liver section of a mouse that received AAV-GFP injection (1×10⁸ viral particles). ˜20% of the liver cells expressed GFP protein, but distribution was not even. FIG. 13B is a graph showing the blood glucose levels of mice receiving portal vein injections of AAV-Ngn3 (triangle line), AAV-Pdx-1-VP16 (diamond line), or AAV-Pdx-1-VP16 and Ngn3 (Square line). A synergistic effect was observed in mice receiving injections of both AAV-Pdx-1-VP16 and Ngn3.

FIG. 14 shows insulin-positive cells in the liver sections of a mouse that received AAV-Pdx1-VP16 (left panel) and of a mouse receiving AAV-Ngn3 (right panel) portal vein injections (1×10⁸ viral particles each). Most of IPCs are distributed under the liver capsule.

FIG. 15 shows that insulin-positive cells are present in liver sections from a mouse that received AAV-PV/AAV-Ngn3 portal vein injection (1×10⁸ viral particles each). Upper left, islet positive control.

FIG. 16 includes four photomicrographs showing glucagon immunostaining in liver sections from a mouse that received AAV-PV/AAV-Ngn3 portal vein injection.

FIGS. 17A and 17B are schematic diagrams showing the sequential expression of pancreatic transcription factors.

FIGS. 18A-18I are amino acid and nucleic acid sequences of pancreatic transcription factors.

FIGS. 19A and 19B show the in vivo kinetics and tissue distribution of rPdx1 following i.p. injection. FIG. 19A is a western blot showing the kinetics of blood Pdx1 pancreas section. FIG. 10B shows a high-power view of insulin-immunostaining in islets of various sizes (small to large). Some show only single insulin+cells.

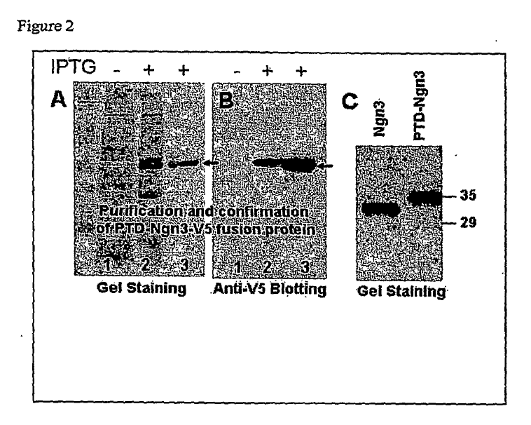

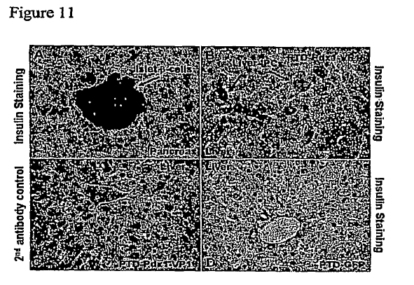

FIGS. 11A-11D are photomicrographs. FIG. 11A shows insulin staining in a pancreatic islet beta cell. FIG. 5B shows that insulin positive cells are present in the liver of a mouse that received intraperitoneal injection of a PTD-Pdx1-VP16 fusion protein. FIG. 2C is a section showing a secondary antibody control, which indicates that the staining is specific. FIG. 2D shows that no insulin staining is present in liver section from a mouse that received intraperitoneal injections of a PTD-GFP control fusion protein.

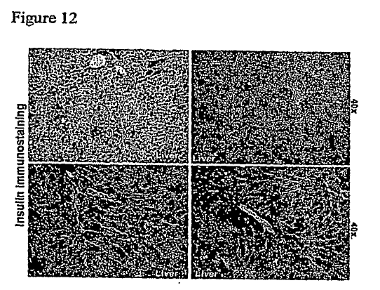

FIG. 12 shows four photomicrographs of insulin-producing cells (IPCs) in liver sections of a mouse that received intraperitoneal injections of a PTD-Pdx1-VP16 and PTD-Ngn3 at day 27 post-injection. Various liver cells producing insulin are present in the liver section (overall 1-2%).

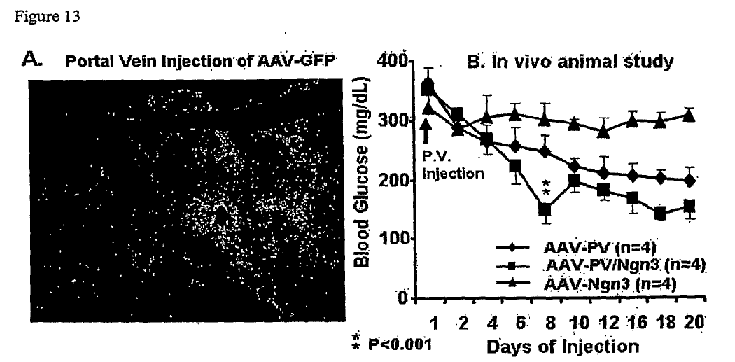

FIGS. 13A and 13B show the effects of AAV-pancreatic transcription factors on blood glucose levels in Stz-induced diabetic mice. FIG. 13A shows GFP protein expression in the liver section of a mouse that received AAV-GFP injection (1×10⁸ viral particles). ˜20% of the liver cells expressed GFP protein, but distribution was not even. FIG. 13B is a graph showing the blood glucose levels of mice receiving portal vein injections of AAV-Ngn3 (triangle line), AAV-Pdx-1-VP16 (diamond line), or AAV-Pdx-1-VP16 and Ngn3 (Square line). A synergistic effect was observed in mice receiving injections of both AAV-Pdx-1-VP16 and Ngn3.

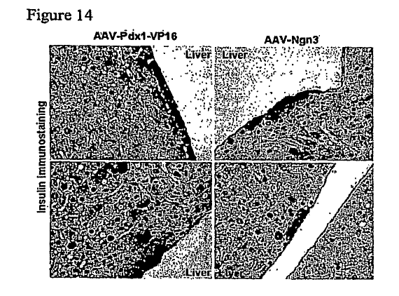

FIG. 14 shows insulin-positive cells in the liver sections of a mouse that received AAV-Pdx1-VP16 (left panel) and of a mouse receiving AAV-Ngn3 (right panel) portal vein injections (1×10⁸ viral particles each). Most of IPCs are distributed under the liver capsule.

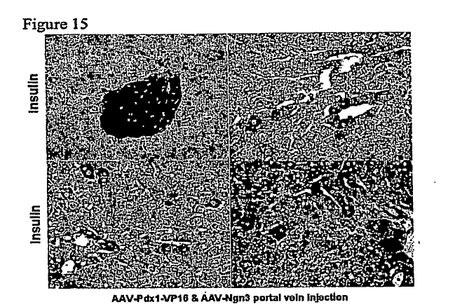

FIG. 15 shows that insulin-positive cells are present in liver sections from a mouse that received AAV-PV/AAV-Ngn3 portal vein injection (1×10⁸ viral particles each). Upper left, islet positive control.

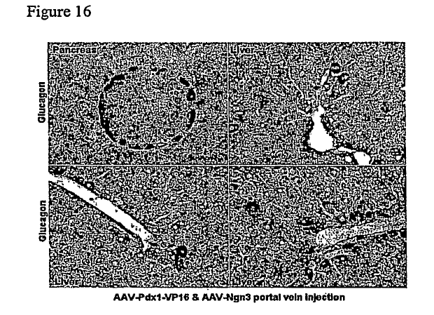

FIG. 16 includes four photomicrographs showing glucagon immunostaining in liver sections from a mouse that received AAV-PV/AAV-Ngn3 portal vein injection.

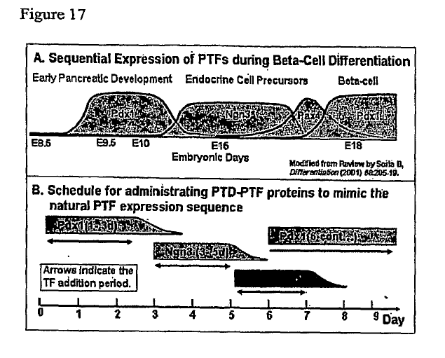

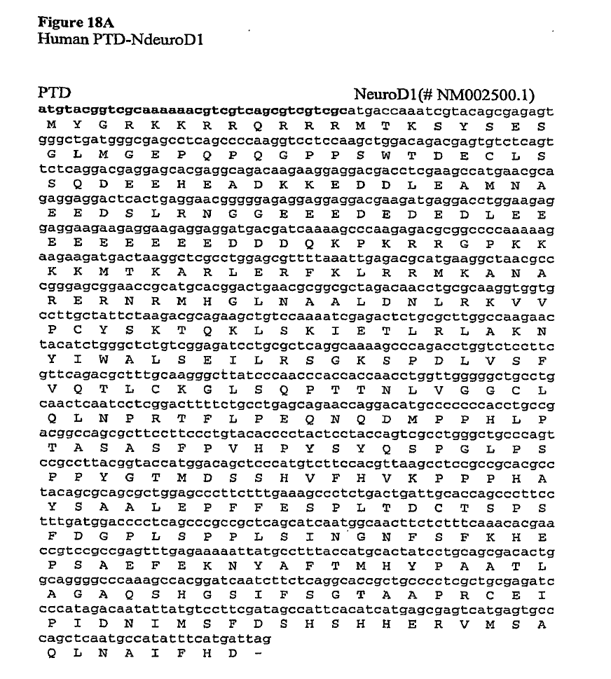

FIGS. 17A and 17B are schematic diagrams showing the sequential expression of pancreatic transcription factors.

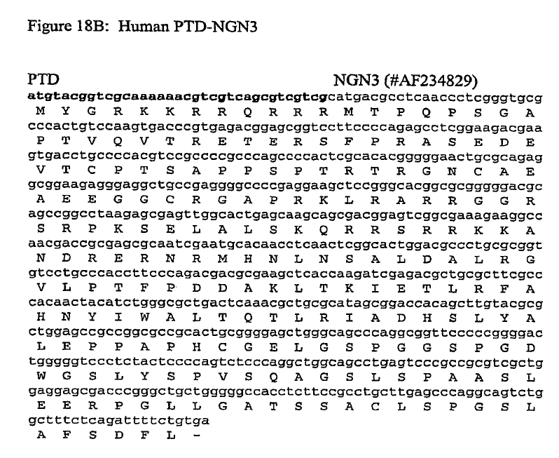

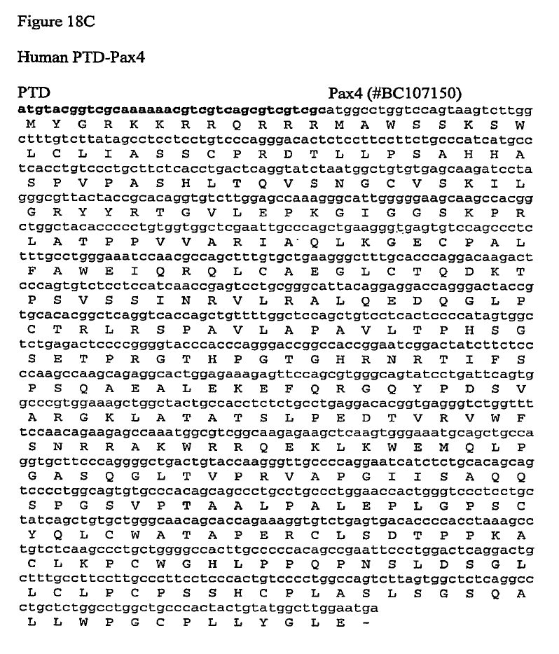

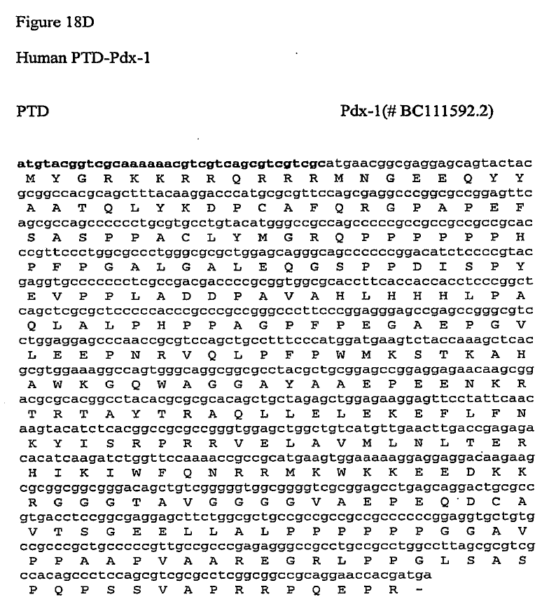

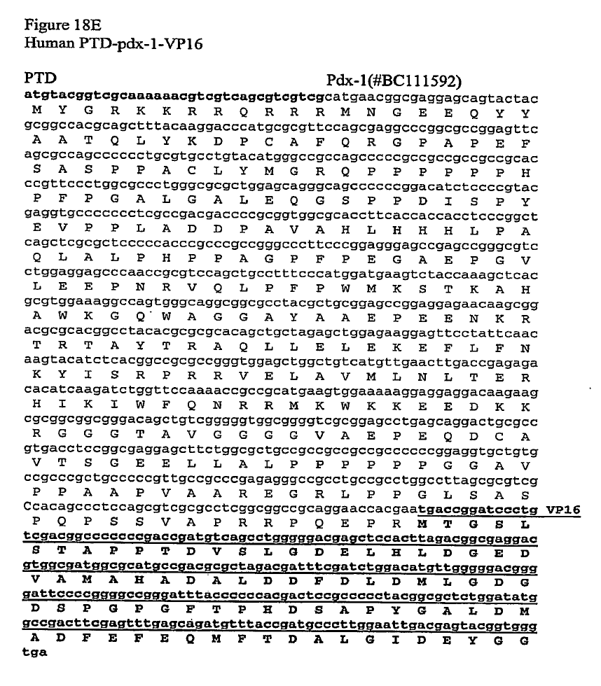

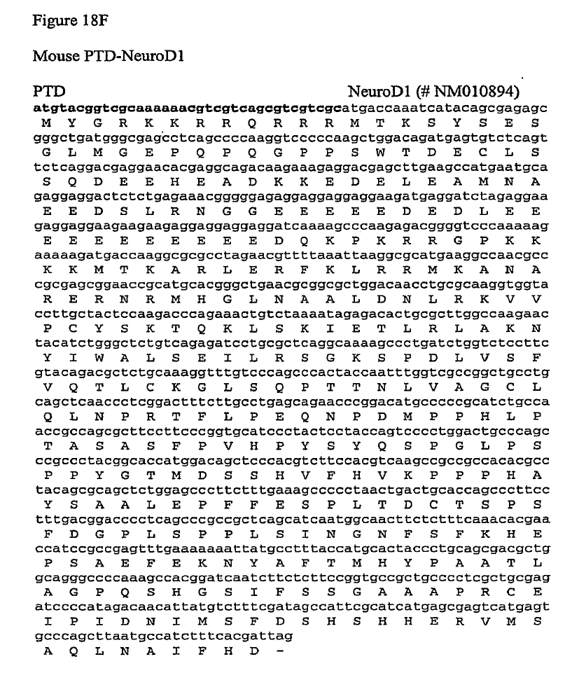

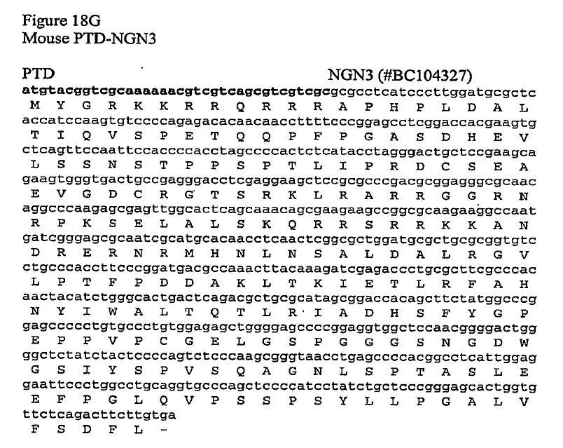

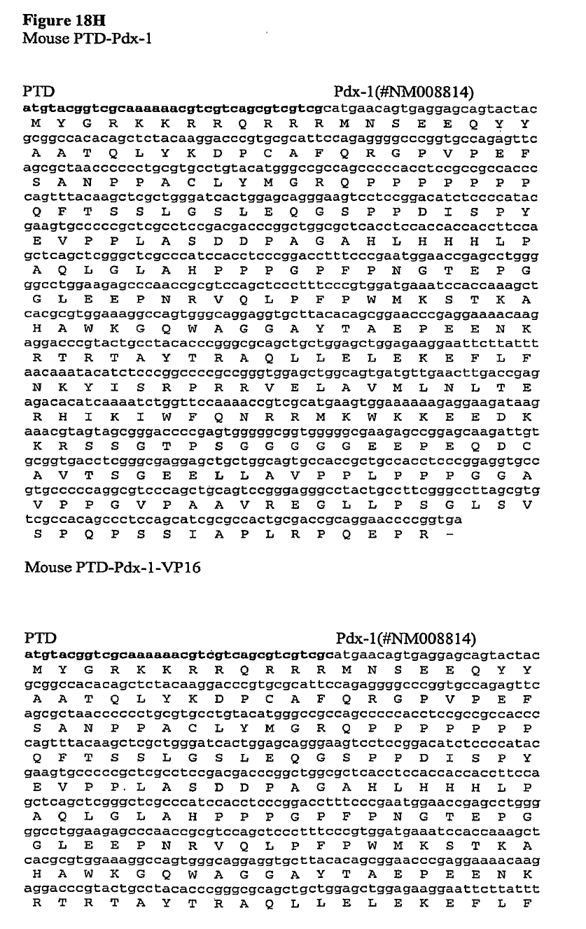

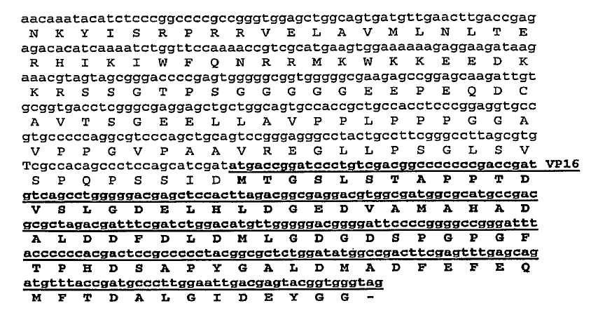

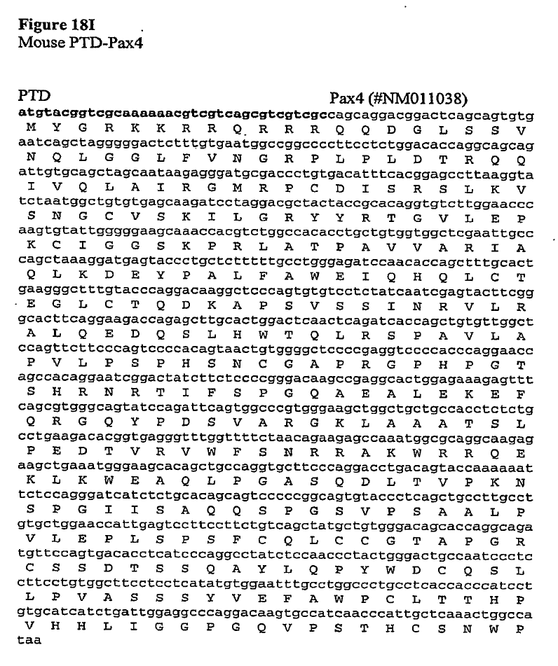

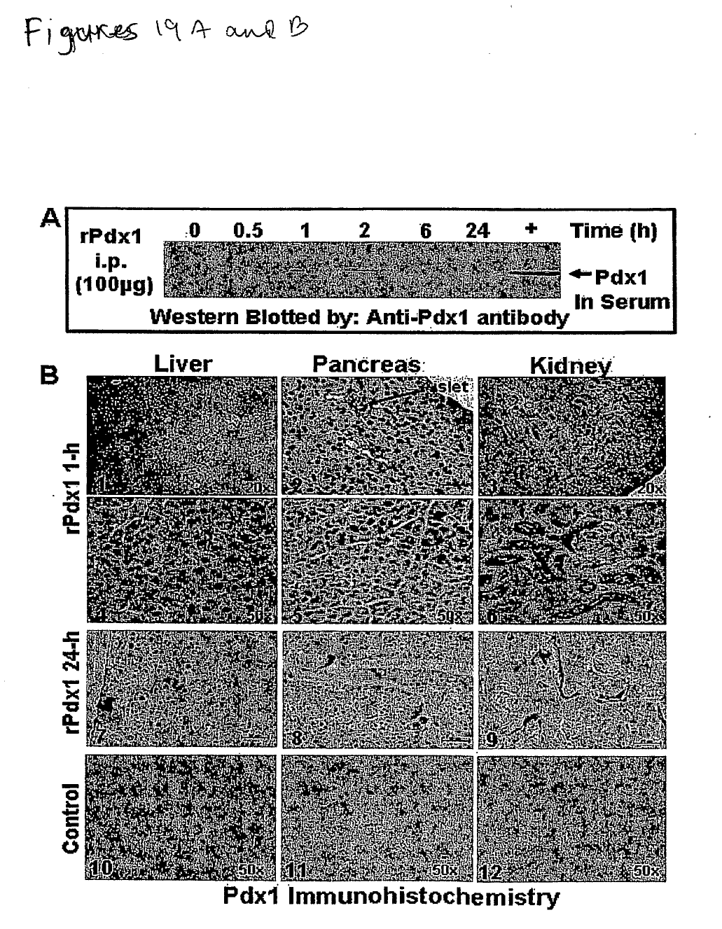

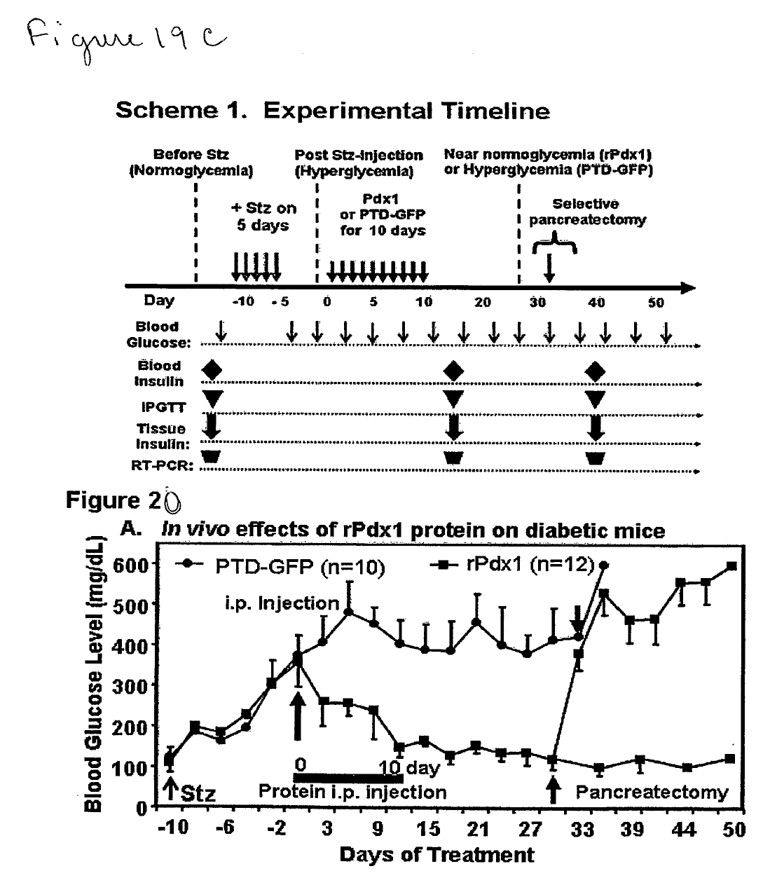

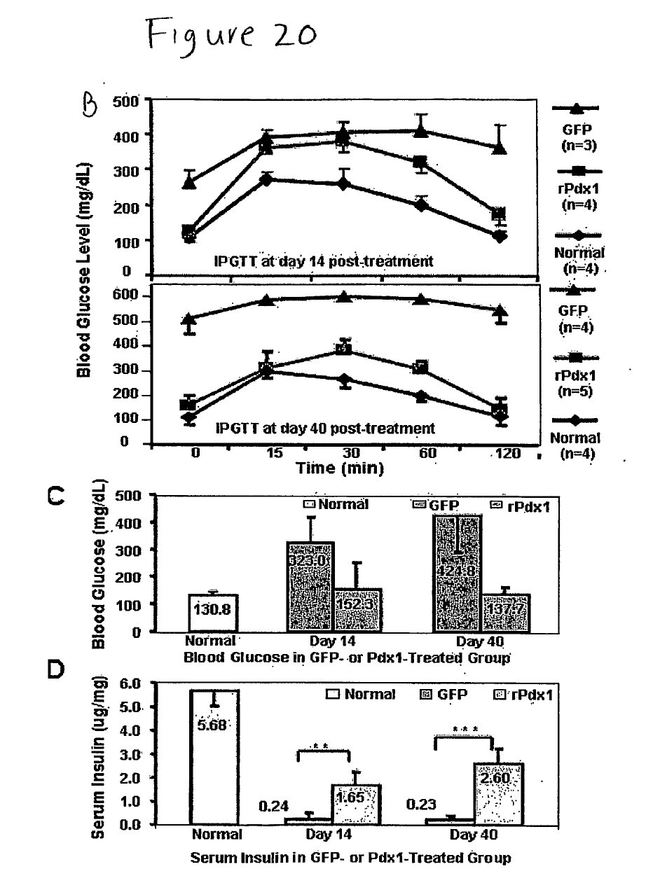

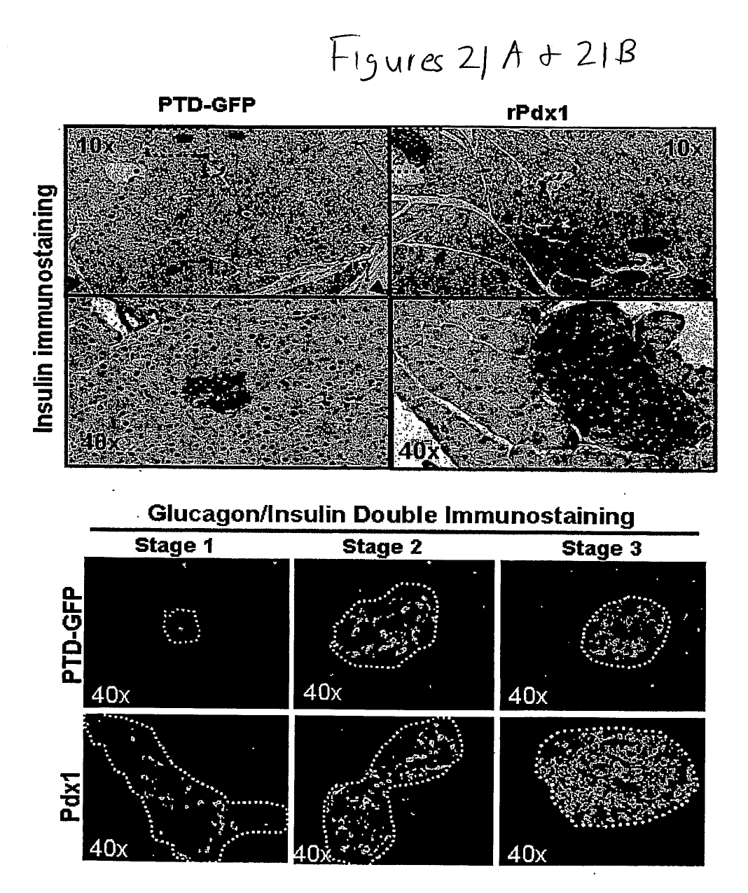

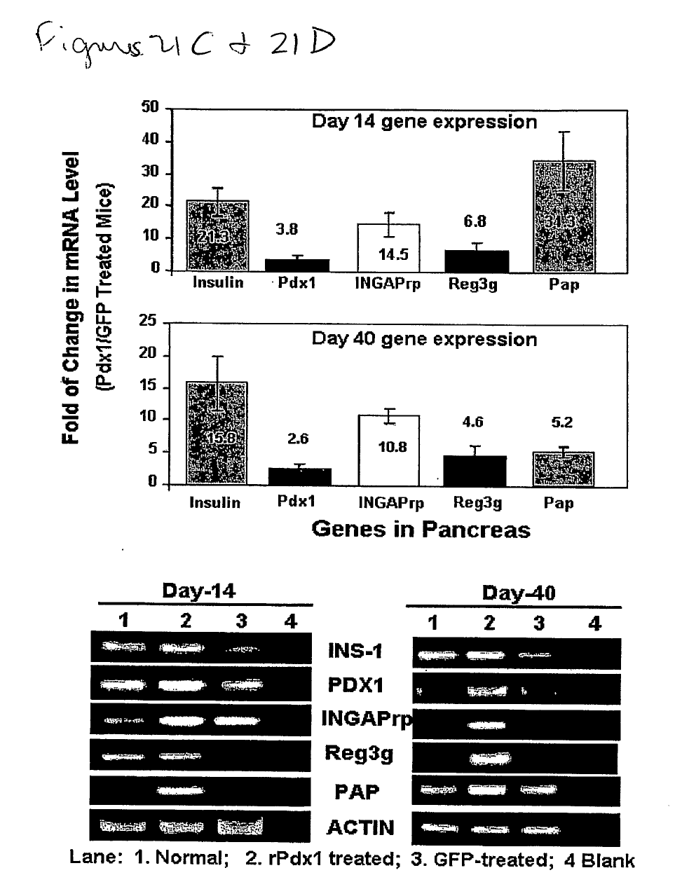

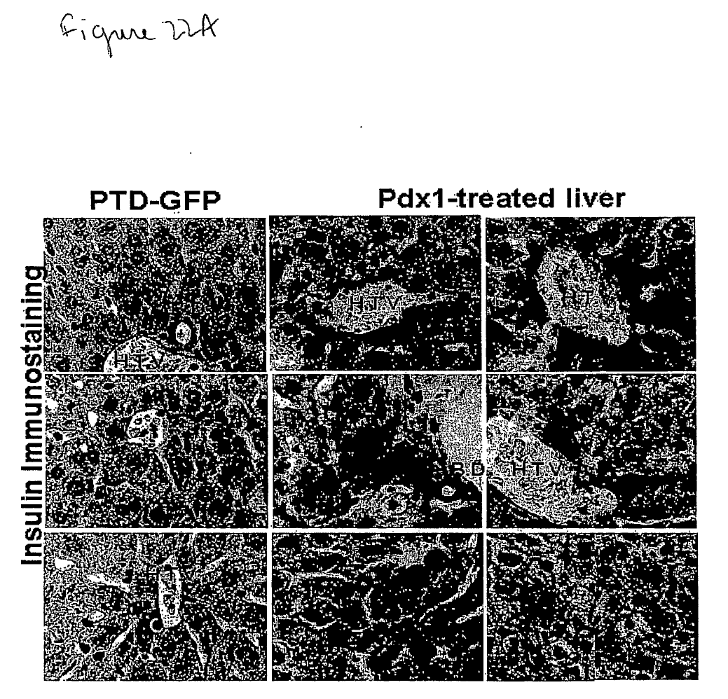

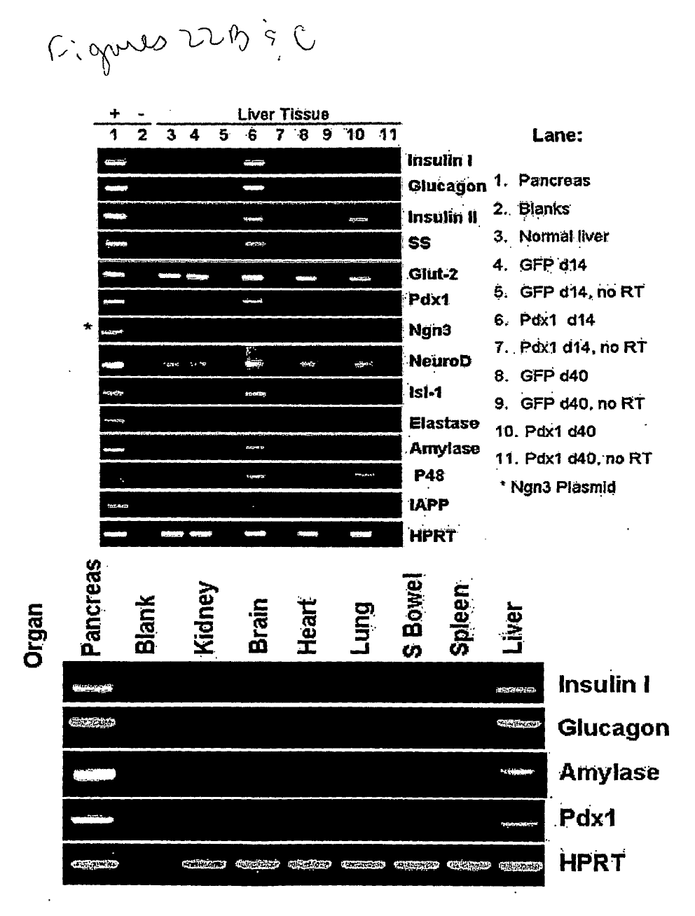

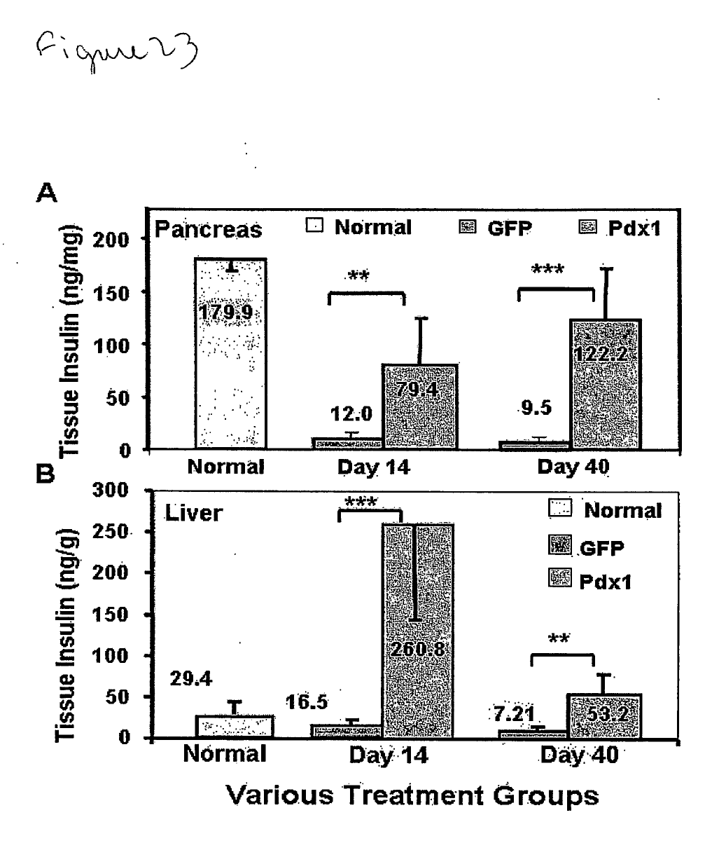

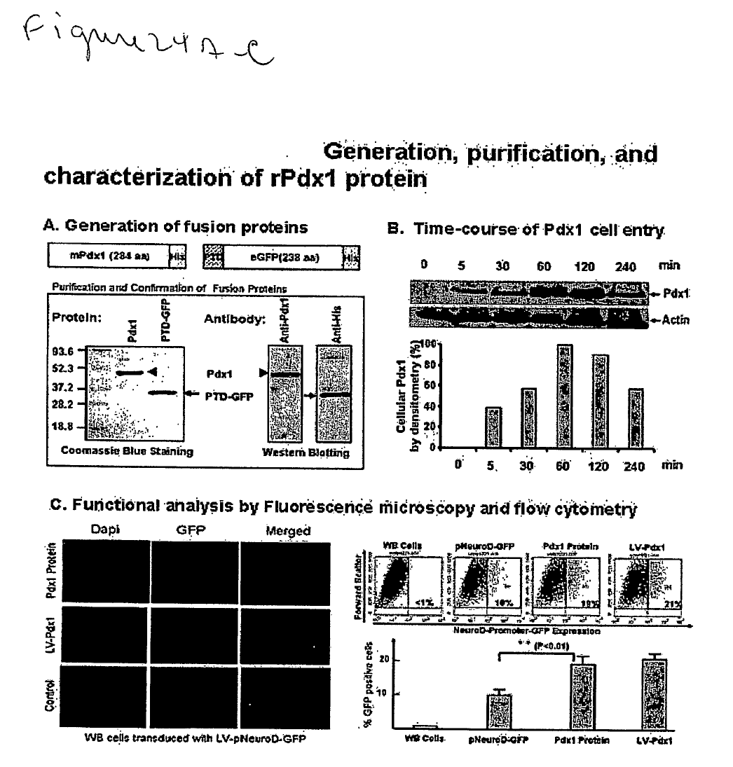

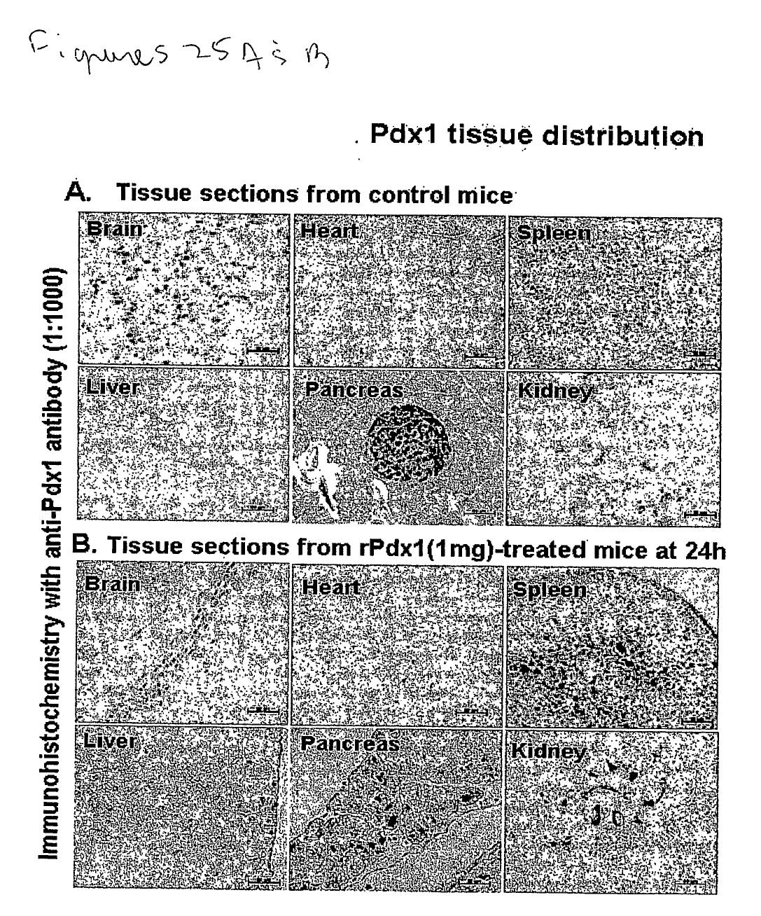

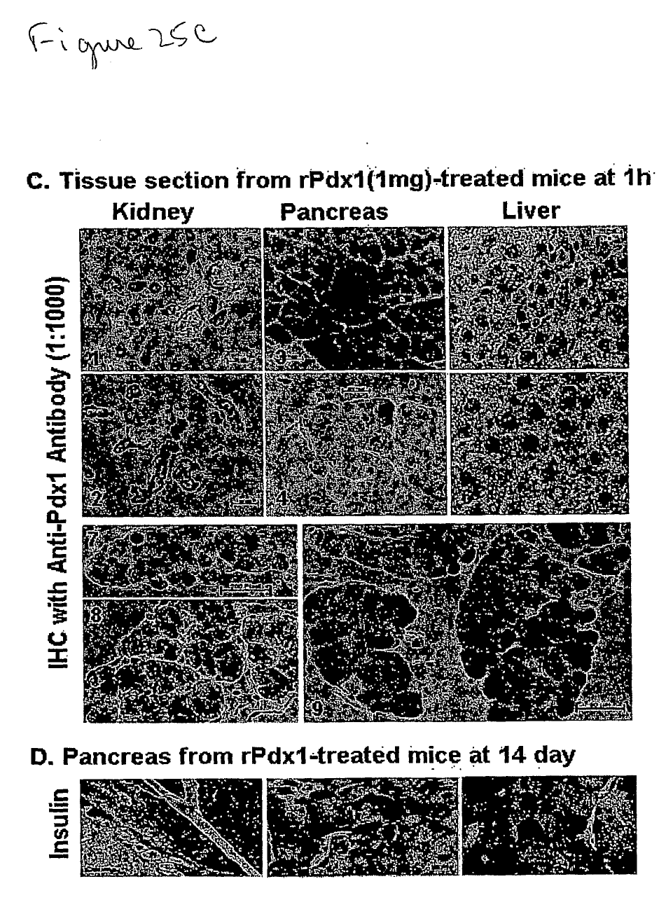

FIGS. 18A-18I are amino acid and nucleic acid sequences of pancreatic transcription factors.

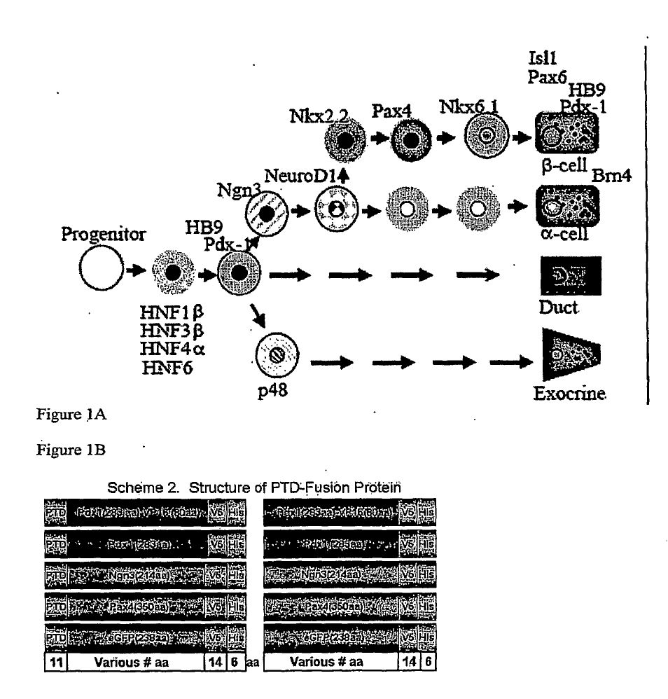

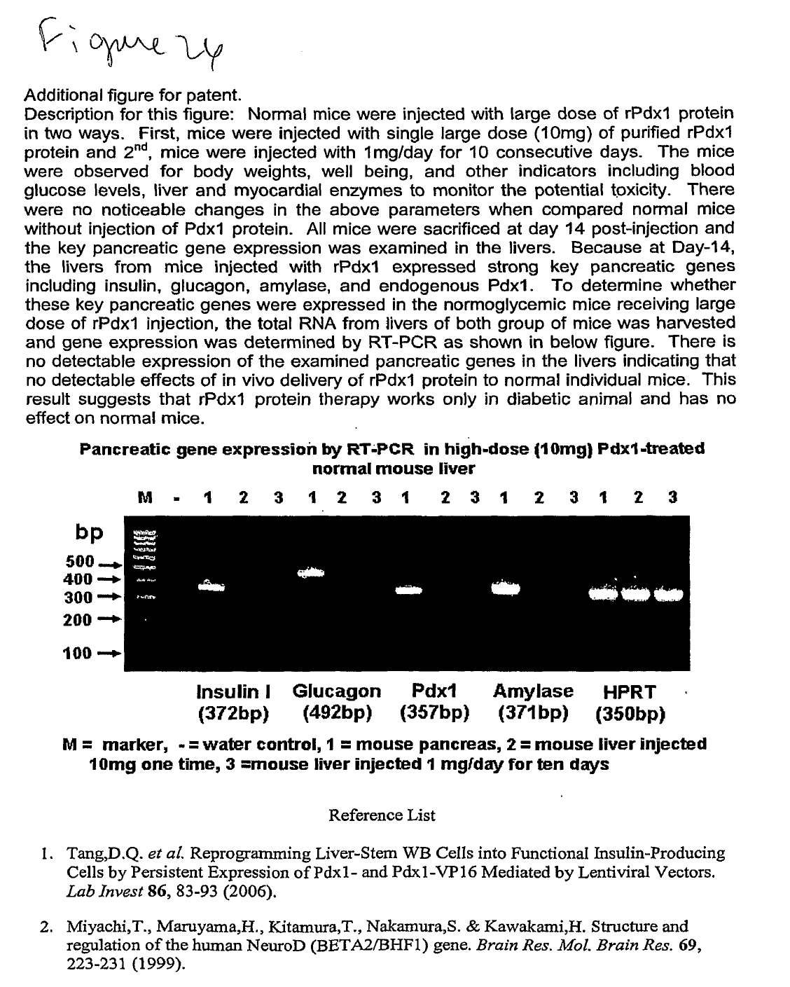

FIGS. 19A and 19B show the in vivo kinetics and tissue distribution of rPdx1 following i.p. injection. FIG. 19A is a western blot showing the kinetics of blood Pdx1 levels. Normal Balb/c mice were i.p. injected with rPdx1 protein (100 μg/mouse). Blood samples were collected at indicated times, and 20 μl serum/lane was loaded in SPDS-PAGE gels. The rPdx1 protein was detected by Western blotting with anti-Pdx1 antibody. FIG. 19B shows twelve panels showing an immunohistochemical analysis of in vivo tissue distribution of Pdx1. Liver, pancreas, and kidney tissues were harvested at 1-hr or 24-hr after rPdx1 (100 μg/mouse) i.p. injection and, fixed in 10% formalin. Paraffin sections were immunostained with anti-Pdx1 antibody (1:2000). Typical distribution patterns of Pdx1 protein in liver (1, 4, and 7), pancreas (2, 5, and 8), and kidney (3, 6, and 9) organs were visualized by light microscopy at 1-hr hour (upper two rows) and 24 hours (lower third row) post-treatment. Pdx1 in of the liver, pancreas, and kidney tissue sections from normal mice is at the bottom row (10-12). The arrow in FIG. 1B-2 indicates a small islet in the pancreas with strong nuclear Pdx1 immunostaining.

FIG. 19C is a schematic diagram showing the experimental timeline used to generate the results described herein. The experimental timeline is outlined here. The baselines of the blood glucose, glucose-challenged insulin release (15 minutes), intraperitoneal glucose tolerance test (IPGTT), and liver and pancreas tissue insulin in normal mice were evaluated first tested. The mice then were then induced to become diabetic by 5× i.p. injection of low dose streptozotocin. Fasting blood glucose levels were monitored regularly and the glucose levels above 250 mg/dL on twice two consecutive readings are were defined as diabetic (hyperglycemic). The glucose-challenged insulin release was measured in some diabetic mice to assess the capacity of residual pancreatic beta-cells in these diabetic mice to handle a glucose surge challenge. Diabetic mice were daily i.p. injected daily with either purified Pdx1 or PTD-GFP fusion protein (100 μg/mouse/injection) for 10 consecutive 10 days and blood glucose levels were monitored as at indicated frequencies. Some mice from both groups were sacrificed around day 14 post-first-dose of protein injection, after an IPGTT measurement, and a blood sample collection at 15 min of IPGTT. The tissues from vital organs were collected and divided into three parts: one part snap-frozen for RT-PCR to examine gene expression; one part fixed in 10% formalin for immunohistological studies; and one part submerged into acid-ethanol for extraction of tissue insulin. Nearly total pancreatectomy was performed in mice with near normoglycemia at day ˜within 30-35 days post-first injection of the fusion proteins and blood glucose levels were monitored. All experimental mice were sacrificed between 40- and 50 days post-protein treatments, and tissues were collected as mentioned above. The similar animal experiments were independently performed six times with variations of sample collections.

FIGS. 20A-20D show the in vivo effects of Pdx1 protein on blood glucose levels. FIG. 20A is a graph showing blood glucose levels in Balb/c mice that were induced to become diabetic (fasting blood glucose levels 250-300 mg/dL on two readings) following 5× low-dose (50 μg/g bw) streptozotocin Stz i.p. injection. Diabetic mice were randomized into experimental rPdx1 or control GFP group and received daily i.p. injection of 100 μg Pdx1 or GFP protein, respectively, for ten consecutive days (red horizontal bar). Blood glucose levels were regularly monitored by tapped the tail vein with a glucometer. Some mice were sacrificed at day 14-15 and day 40 post-first injection. IPGTT was performed in normal, GFP-, or rPdx1-treated mice around day 14 and Day 40 post-first injection. Nearly total pancreatectomy was performed in selected control and rPdx1-treated mice around day 30 post-treatment. FIG. 20B provides two graphs showing the results of an intraperitoneal glucose tolerance test (IPGTT). Mice were fasted for at least 8-hrs before IPGTT. A bolus dose of glucose (1 mg/g bw) was i.p. injected and blood glucose was measured at 0, 15-minutes, 30-minutes, 60-minutes, and 120-minutes in normal (bottom black line), rPdx1-(middle gray line)-, or GFP-(top gray line)-treated mice. FIG. 20C is a graph showing blood glucose levels and FIG. 20D shows insulin levels following IPGTT at 15-minutes at Day-14 and Day-40 post-treatment. Mouse blood glucose levels were monitored as mentioned above. Blood samples were collected from normal and treated mice challenged by i.p. injection of a bolus dose of glucose (1 mg/g bw) at 15-minutes. Insulin levels were assayed by a mouse ultra-sensitive insulin ELISA kit. Each group contains at least five mice.

FIG. 21A-21D shows that Pdx1 protein promotes pancreatic islet cell regeneration. FIG. 21A provides four panels showing insulin immunohistochemistry. Sections from paraffin embedded pancreas tissues from mice treated with either GFP (left) or rPdx1 (right) were immunostained with anti-insulin antibody (1:1000). Representative images were taken at 10× (upper panel) or 40× (lower panel) magnification. FIG. 21B shows insulin/glucagon double-immunostaining of pancreatic tissue. Paraffin sections from GFP- and rPdx1-treated mouse pancreas tissue were immunostained with both rabbit-anti-glucagon/PE (red) and Guinea pig-anti-insulin/FITC (green) and visualized under fluorescence microscopy. All images were taken at 40× magnification. FIG. 21C provides two graphs showing quantitative real-time RT-PCR analyses. Total RNA collected from pancreas tissues of the diabetic mice at day-14 and day-40 post-rPdx1- or GFP-treatment (100 μg/day for 10 days), and expression of the five target genes including insulin, Pdx1, INGAPrP, Reg3≢5, and PAP were examined by real-time RT-PCR. Expression levels were corrected normalized relative to the expression of the of β-actin gene. Results represent at least three individual mice in each group. Fold difference of relative gene expression was calculated as the ratio of the mean C_{T}-values of target gene expression (compared with standard actin cDNA) in rPdx1-treated pancreas to/the mean C_{T}-values of these target genes (compared with standard actin cDNA) in GFP-treated pancreas samples. Abbreviations: INGAPrP, =islet neogenesis-associated protein related protein,; Reg3≢5, =regenerating islet-derived 3 gamma,; PAP, =pancreatitis-associated protein. FIG. 21D is an agarose gel showing real-time PCR bands in agarose gels. Real-time PCR products were run in agarose gels to confirm the size and specificity (single band in the gels) on day-14 and day-40 post-treatment samples.

FIGS. 22A-22C show that Pdx1 protein promotes liver cell transdifferentiation into insulin-producing cells. FIG. 22A includes nine panels showing insulin immunohistochemical staining. Paraffin sections from livers were incubated with anti-insulin antibody (1:250) overnight at 4° C. Photo images were taken using 40× objective lenses. Insulin-positive cells were observed in the rPdx1-treated mouse liver section at day 14 post-treatment. Abbreviations: B.D.=−bile duct, H.T.V.=−hepatic terminal vein. B; black arrows indicate condensed nuclear chromatin feature of single bi-nucleated insulin-expressing liver cells. FIG. 22B shows the expression of pancreatic genes in the livers. Reverse transcription-polymerase chain reaction (RT-PCR) amplifications of RNAs extracted from livers of normal, GFP-, or rPdx1-treated mice were analyzed by agarose gel electrophoresis. RNA isolated from mouse pancreas was used as a positive control. For Ngn3 RT-PCR analysis, Ngn3 cDNA plasmid (*) was used as positive control because adult pancreas does not express this gene. Abbreviation: No RT=−no reverse transcription, D114- or D40=−day 14 or day 40 post-first-protein injection. FIGS. 22C and 22D are agarose gels showing the expression of pancreatic genes in other organs. Total RNA collected from other organ tissues of the rPdx1-treated diabetic mice at day-14 post-treatment and expression of four key pancreatic genes (PDX1, insulin, glucagon, and amylase) were examined by RT-PCR. All RT-PCR results represent at least three individual mice in each particular group and the results were independently repeated three times.

FIGS. 23A and 23B are graphs showing pancreas and liver tissue insulin measurements. FIG. 23A shows pancreatic tissue insulin measurement. A separate group of normal (n=4) or diabetic mice treated with either GFP (n=4) or rPdx1 (n=5) via i.p. injection for 10 consecutive days was used for this study. Mice were killed sacrificed at day-14 or day-40 post-injection and the entire liver or pancreas was weighed and collected for extraction of tissue insulin to prevent reduce sampling variations. Tissue insulin was extracted in acid-ethanol and determined by ELISA with a mouse ultra-sensitive insulin ELISA kit. Tissue insulin content in the pancreas was expressed as the amount of insulin (ng) per milligram wet weight of pancreatic tissue. **=(p<0.05) and ***=(p<0.001). FIG. 23B shows liver tissue insulin measurement. Liver tissue insulin was extracted using the same methods as described above. Tissue insulin content in the liver was expressed as the amount of insulin (ng) per gram wet weight of liver tissue. A day-14, the liver insulin content is significantly higher in the rPdx1-treated (n=6) mice at day 14 than the livers in normal (n=4), or GFP-treated (n=5) mice. **=(p<0.05). ***=(p<0.001).

FIG. 24A-24C shows the cloning, expression, purification, and characterization of mouse Pdx1 and PTD-GFP fusion proteins. FIG. 24A shows the generation of fusion proteins. The top panel represents schematic structures of fusion proteins of mouse Pdx1 and PTD-GFP. The gray box represents the antennapedia-like protein transduction domain in the Pdx1 protein. The cDNA fragments coding mouse Pdx1 or PTD-GFP were cloned into the expression plasmid. Proteins were expressed and purified by a Ni-column. The bottom panel showed purified proteins in a 10% SDS-PAGE gel and was stained with Coomassie Brilliant Blue R solution (left panel). Lane 1 represents molecular weight markers; lane 2, Pdx1; and lane 3, PTD-GFP. The Right panel is confirmation of the fusion proteins by Western blotting using anti-Pdx1 antibody (lane 1) and anti-his-tag antibody (lane 2). The rPdx1 and PTD-GFP fusion proteins are confirmed as indicated by arrowheads or arrows, respectively. FIG. 24B shows the time-course of cell entry of Pdx1 protein. WB cells were incubated with Pdx1 (1 μM) for indicated times and washed three times with PBS. Cell lysates were separated and probed by Western blotting with rabbit anti-Pdx1 (1:1000) or anti-actin (1:5000) antibody. (B) The relative amount of cellular Pdx1 protein was quantified by densitometry. Bands were scanned for obtaining their density values, and the values were standardized with their corresponding house keeping protein actin. The peak reading is defined as 100%, and the rest of the values are divided by the highest reading to give the comparison of relative amount of cellular Pdx1 protein. FIG. 24C shows a functional analysis of rPdx1 protein. WB cells were transduced by LV-pNeuroD-GFP reporter gene. The WB cells expressing pNeuroD-GFP-expressing reporter gene WB cells were visualized and quantified at 72-hrs hours post-treatments with either Pdx1 protein or LV-Pdx1 by fluorescence microscopy and flow cytometric analysis. Left panels show fluorescence images of pNeuroD-GFP-expressing cells on cytospin slides. Right-upper panel shows flow dot-plot. Ls; and lower panel is a histogram showing percentage of GFP-expressing cells. Results were representative of three independent experiments.

FIGS. 25A-25D show Pdx1 tissue distribution. Normal Balb/c mice were i.p. injected with rPdx1 protein (1 mg) and sacrificed at 1-hour or 24-hour post-injection. Tissue sections from normal or rPdx1-injected mice were immunostained with anti-Pdx1 antibody (1:1000, made in our lab against rPdx1 protein). FIG. 25A shows six tissue sections from control mice. There is background nuclear staining in brain tissue and pancreatic islet shows positive nuclear staining of Pdx1 protein. FIG. 25B shows six tissue sections from rPdx1-treated mice at 24-hrs post-injection. At 24-hours post-injection, a minimal amount of rPdx1 protein was detected in the liver, pancreas, and kidney sections. Sections from brain, heart, and spleen show a background staining similar to normal tissue without rPdx1 injection. FIG. 25C includes nine panels showing tissue sections from rPdx1-treated (1 mg) mice at 1-hour post-injection. Strong Pdx1 immuno-reactivity was detected in caps and proximal tubular cells on the section of kidney (1-2), in nuclei and cytoplasm in exocrine pancreatic acinar cells on pancreas section (3-4, 7-9), and in the nuclei of hepatocytes on the liver section (5-6). FIG. 25D includes three panels showing insulin-positive cells among exocrine pancreatic acinar cells in Pdx1-treated diabetic mice at day-14 post-injection.

FIG. 26 is an agarose gel showing the expression of insulin I, glucagon, Pdx1, amylase, and horse radish peroxidase in normal mouse liver.

### DETAILED DESCRIPTION OF THE INVENTION

The present invention generally provides therapeutic compositions and methods for treating a disease, disorder, or injury characterized by a deficiency in the number or biological activity of a cell of interest. In one embodiment, the method provides protein compositions (e.g., protein transduction domain-containing fusion polypeptides) for generating reprogrammed cells (e.g., adult cells or embryonic stem cells), or for increasing regeneration in a cell, tissue, or organ of interest. If desired, these compositions are administered in combination with therapeutic nucleic acid molecules that provide for the persistent expression of a therapeutic polypeptide of the invention. Such methods are useful for treating subjects having a deficiency in a particular cell type or in a polypeptide produced by that cell type. The invention is based, at least in part, on the observation that insulin producing cells can be generated by reprogramming adult liver cells to insulin producing cells by contacting the liver cells with a pancreatic transcription factor fused to a protein transduction domain. In addition, the invention provides compositions and methods that induce pancreatic cell regeneration. Accordingly, the invention also provides prophylactic and therapeutic methods and compositions for ameliorating or preventing hyperglycemia associated with type 1 and type II diabetes and related complications.

### Pancreatic Transcription Factors

The differentiation and maturation of endocrine islet cells during embryonic development is a complex process that is controlled by a unique pattern of gene regulation (see FIG. 1A). Numerous pancreatic transcription factors (PTFs) are known to play important roles in specifying the different cell types found within the pancreas. Of these transcription factors, Pancreatic and Duodenal Homeobox gene-1 (Pdx-1) has the greatest likelihood for encoding key proteins that distinguish liver and pancreas. During embryogenesis, Pdx-1, which is expressed in all progenitor cells differentiating toward the exocrine and endocrine pancreas (Soria Differentiation 2001; 68(4-5):205-219; Hui et al., Eur J Endocrinol 2002; 146(2):129-141) plays an essential role in normal pancreas development. Indeed, no pancreatic tissue exists in Pdx-1 knockout mice. The lack of a functional Pdx-1 protein in humans results in agenesis of the pancreas. In adults, Pdx-1 expression is restricted to β-cells and about 20% of δ-cells, where it plays a key role in insulin gene expression (Soria Differentiation 2001; 68(4-5):205-219; Hui et al., Eur J Endocrinol 2002; 146(2):129-141).

Other PTFs are expressed selectively in the endocrine cells in the developing pancreas where they may play a role in endocrine cell fate decisions. These pancreatic transcription factors contain homeodomains and can be divided into early factors, including neurogenein 3 (Ngn3), Nkx2.2, and Nkx6.1, that are coexpressed in endocrine progenitor cells and later factors (Pax4, Pax6, and Isl-1) that found in more mature cells (Soria Differentiation 2001; 68(4-5):205-219; Wilson et al., Mech Dev 2003; 120(1):65-80). The basic helix-loop-helix (bHLH) transcription factor Ngn3 is transiently expressed in endocrine progenitor cells during pancreas development and directly regulates Beta2/NeuroD. Ngn3 controls endocrine cell fate decisions in multipotent pancreatic endodermal progenitors (Gradwohl et al., Proc Natl Acad Sci USA 2000; 97(4):1607-1611; Gu et al., Development 2002; 129(10):2447-2457; Schwitzgebel et al., Development 2000; 127(16):3533-3542). Beta2/NeuroD is a bHLH protein Beta2/NeuroD that is a direct downstream target gene of Ngn3. Beta2/NeuroD is expressed in pancreatic endocrine cells and activates insulin gene transcription. Pax4 and Pax6, are two homeodomain proteins expressed both in the developing gut and in the adult pancreas, where they function in the specification of different cell types. Pax4 is a key factor in the late-stage differentiation of insulin-producing β cells and somatostatin producing δ cells (Sosa-Pineda et al., Nature 1997; 386(6623):399-402). Pax4 is transiently expressed in δ developing β cells and is shut down by autoregulation (Sosa-Pineda et al., Nature 1997; 386(6623):399-402). Transfection of mouse embryonic stem cells with Pax4 leads to marked increases in IPCs compared with Pdx-1 transfected cells (Blyszczuk et al., Proc Natl Acad Sci USA 2003; 100(3):998-1003). In contrast, Pax6 is required in the generation of glucagon producing a cells (St Onge et al., Nature 1997; 387(6630:406-409). Nkx2.2 and Nkx6.1 function in the development of pancreatic β cells and they have a similar pattern of gene expression (Sander et al., Genes Dev 1997; 11(13):1662-1673). Isl-1 is required for the differentiation of islet cells (Ahlgren et al., Nature 1997; 385(6613):257-260) because no endocrine cells are present in Isl-1 knockout mice.

Because pancreatic transcription factors such as Pdx1 and Ngn3 exert upstream control over the commitment of stem or progenitor cells to differentiate into pancreatic endocrine cells (Ahlgren et al., Nature 1997; 385(6613):257-260) and Pax4 exerts a second-wave of commitment of the endocrine precursor cells to islet beta cells, these pancreatic transcription factors are useful for reprogramming cells (e.g., adult cells or embryonic stem cells). In one embodiment, an adult cell that fails to express insulin is converted into an insulin-producing cell. For example, as reported herein, pancreatic transcription factors are used to generate insulin-producing cells from liver-derived cells or liver stem cells. Transcription factors were commonly thought of as cytosolic proteins without the ability to translocate from one cell to another. More recently, evidence indicates that some transcription factors behave as paracrine signaling molecules. Such transcription factors typically include a protein transduction domain. It was recently reported that the PDX-1 protein contains an antennapedia-like protein transduction domain that can transduce pancreatic duct and islet cells (Noguchi et al., Diabetes 2003; 52(7):1732-1737). Protein-engineering can be used to provide other transcription factors that include one or more protein transduction domains.

### Protein Transduction Domains

Protein transduction domains are short peptide sequences that enable proteins to translocate across the cell and nuclear membranes, leading to entry into the cytosol by means of atypical secretory and internalization pathways (Joliot et al., Nat Cell Biol 2004; 6(3):189-196). In 1988 Green and Loewenstein discovered that the human immunodeficiency virus type 1 (HIV-1) TAT-protein, an 86-amino acid protein, could rapidly enter cells and was even capable of entering the cell nucleus (Green and Loewenstein P M. Cell 1988; 55(6):1179-1188). Building on this observation, a minimal unidecapeptide protein transduction domain (corresponding to residues 47-57 of HIV-1 TAT) was developed by Dowdy and co-workers (Schwarze et al., Science 1999; 285(5433):1569-1572). This unidecapeptide sequence was used successfully to deliver an NH2-terminal TAT-β-galactosidase fusion protein (120 kDa) to mouse tissues via intraperitoneal injections into mice (Schwarze et al., Science 1999; 285(5433):1569-1572). The TAT-β-galactosidase fusion protein retained biological activity. This general method has now been successfully used for the transduction of a variety of proteins. PTD-containing peptides or proteins are taken up by cells within 5 minutes at concentrations as low as 100 nM as assessed by direct labeling with fluorescence or by indirect immunofluorescence using antibodies. This uptake is independent of endocytotic mechanisms, transmembrane protein channels, and protein receptor binding. In addition, in vitro studies have demonstrated that protein transduction domain-mediated translocation occurs at low temperatures and exhibits no strong cellular specificity.

### Recombinant Polypeptide Expression

The invention generally provides protein-based therapies useful for treating diabetes and other diseases, disorders, or injuries associated with a deficiency in the number or biological activity of a cell of interest. In general, a protein-based therapeutic comprises a transcription factor operably linked to a protein transduction domain, where the protein transduction domain is capable of acting as a “molecular passport” to permit entry into cells of a biologically active transcription factor. The transcription factor acts to reprogram the cell. The reprogrammed cell has an altered transcriptional and/or translational profile, i.e., expresses an altered set of mRNAs and/or polypeptides expressed relative to an untreated control cell.

As described in more detail below, virtually any transcription factors of interest can be fused to a protein transduction domain and used for protein therapy. Advantageously, such fusion proteins can be delivered to cells in vitro or in vivo. In one embodiment, a fusion protein of the invention is used to contact a cell in vitro, such that the cell takes up the fusion protein. The cell is subsequently delivered to a subject for a therapeutic purpose. Alternatively, a fusion protein of the invention is administered to a cell, tissue, or organ in situ, such that the cell, tissue, or organ takes up the fusion protein to achieve a therapeutic purpose. In one working embodiment, a fusion protein of the invention is a pancreatic transcription factor operably linked to a protein transduction domain. When this fusion protein contacts a hepatocyte, hepatic stem cell, or other somatic cell (e.g., pancreatic progenitor cell, pancreatic stem cell, islet cell, endocrine cell, or exocrine cell) it reprograms the cell to produce insulin and/or glucagon. In another embodiment, a pancreatic transcription factor fused to a protein transduction domain increases the regenerative capacity of a pancreatic cell (e.g., pancreatic progenitor cell, pancreatic stem cell, islet cell, endocrine cell, or exocrine cell). This increase in regenerative capacity typically results in an increase in the production of insulin, such that normoglycemia is restored in a hyperglycemic subject. Desirably, insulin production is increased by at least about 1, 2, 3, 4, 5 times, or by at least about 10, 12, 15 or 20 times relative to a reference amount (e.g., the amount produced prior to treatment or produced in an untreated control).

Recombinant fusion polypeptides of the invention are produced using virtually any method known to the skilled artisan. Typically, recombinant polypeptides are produced by transformation of a suitable host cell with all or part of a polypeptide-encoding nucleic acid molecule or fragment thereof in a suitable expression vehicle. Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., Current Protocol in Molecular Biology, New York: John Wiley and Sons, 1997). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

A variety of expression systems exist for the production of the polypeptides of the invention. Expression vectors useful for producing such polypeptides include, without limitation, chromosomal, episomal, and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof.

One particular bacterial expression system for polypeptide production is the E. coli pET expression system (e.g., pET-28) (Novagen, Inc., Madison, Wis). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein.

Another bacterial expression system for polypeptide production is the pGEX expression system (Pharmacia). This system employs a GST gene fusion system that is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Alternatively, recombinant polypeptides of the invention are expressed in Pichia pastoris, a methylotrophic yeast. Pichia is capable of metabolizing methanol as the sole carbon source. The first step in the metabolism of methanol is the oxidation of methanol to formaldehyde by the enzyme, alcohol oxidase. Expression of this enzyme, which is coded for by the AOX1 gene is induced by methanol. The AOX1 promoter can be used for inducible polypeptide expression or the GAP promoter for constitutive expression of a gene of interest.

Once the recombinant polypeptide of the invention is expressed, it is isolated, for example, using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra). Alternatively, the polypeptide is isolated using a sequence tag, such as a hexahistidine tag, that binds to nickel column.

Once isolated, the recombinant protein can, if desired, be further purified, e.g., by high performance liquid chromatography (see, e.g., Fisher, Laboratory Techniques In Biochemistry and Molecular Biology, eds., Work and Burdon, Elsevier, 1980). Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). These general techniques of polypeptide expression and purification can also be used to produce and isolate useful peptide fragments or analogs (described herein).

### Transcription Factor/Protein Transduction Domain Fusion Polypeptides and Analogs

Also included in the invention are transcription factor/protein transduction domain fusion polypeptides or fragments thereof that are modified in ways that enhance their ability to reprogram a cell or their ability to induce regeneration. In other embodiments, variations in the sequence increase protein solubility or yield. For example, the invention provides a modified pancreatic transcription factor fusion protein having an enhanced ability to reprogram a liver-derived cell to an insulin-producing cell. In other examples, the modified pancreatic transcription factor fusion protein increases the regenerative capacity of a pancreatic cell. Alternatively, the alteration is in the protein transduction domain, and the altered domain increases transport of an operably linked protein into a cell or cellular compartment, such as the nucleus. In other embodiments, the alteration in the protein transduction domain reduces interference with a biological activity of an operably linked polypeptide.

The invention provides methods for optimizing a transcription factor or protein transduction domain amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also provides fragments of any one of the polypeptides or peptide domains of the invention. As used herein, the term “a fragment” means at least 5, 10, 13, or 15 amino acids. In other embodiments a fragment is at least 20 contiguous amino acids, at least 30 contiguous amino acids, or at least 50 contiguous amino acids, and in other embodiments at least 60 to 80, 100, 200, 300 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Non-protein transcription factor/protein transduction domain fusion analogs have a chemical structure designed to mimic the fusion proteins functional activity. Such analogs are administered according to methods of the invention. Fusion protein analogs may exceed the physiological activity of the original fusion polypeptide. Methods of analog design are well known in the art, and synthesis of analogs can be carried out according to such methods by modifying the chemical structures such that the resultant analogs increase the reprogramming or regenerative activity of a reference transcription factor/protein transduction domain fusion polypeptide. These chemical modifications include, but are not limited to, substituting alternative R groups and varying the degree of saturation at specific carbon atoms of a reference fusion polypeptide. Preferably, the fusion protein analogs are relatively resistant to in vivo degradation, resulting in a more prolonged therapeutic effect upon administration. Assays for measuring functional activity include, but are not limited to, those described in the Examples below.

### Test Compounds and Extracts

In general, fusion polypeptides having reprogramming activity or regeneration inducing activity are identified from large libraries of natural product or synthetic (or semi-synthetic) extracts or chemical libraries or from polypeptide or nucleic acid libraries, according to methods known in the art. Such candidate polypeptides or the nucleic acid molecules encoding them may be modified to include a protein transduction domain. The modified polypeptides are then screened for the desired activity. Those skilled in the field of drug discovery and development will understand that the precise source of test extracts or compounds is not critical to the screening procedure(s) of the invention. Agents used in screens may include known compounds (for example, known polypeptide therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as the modification of existing polypeptides.

Libraries of natural polypeptides in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). Such polypeptides can be modified to include a protein transduction domain using methods known in the art and described herein. In addition, natural and synthetically produced libraries are produced, if desired, according to methods known in the art, e.g., by standard extraction and fractionation methods. Examples of methods for the synthesis of molecular libraries can be found in the art, for example in: DeWitt et al., Proc. Natl. Acad. Sci. U.S.A. 90:6909, 1993; Erb et al., Proc. Natl. Acad. USA 91:11422, 1994; Zuckermann et al., J. Med. Chem. 37:2678, 1994; Cho et al., Science 261:1303, 1993; Carrell et al., Angew. Chem. Int. Ed. Engl. 33:2059, 1994; Carell et al., Angew. Chem. Int. Ed. Engl. 33:2061, 1994; and Gallop et al., J. Med. Chem. 37:1233, 1994. Furthermore, if desired, any library or compound is readily modified using standard chemical, physical, or biochemical methods.

Numerous methods are also available for generating random or directed synthesis (e.g., semi-synthesis or total synthesis) of any number of polypeptides, chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, chemical compounds to be used as candidate compounds can be synthesized from readily available starting materials using standard synthetic techniques and methodologies known to those of ordinary skill in the art. Synthetic chemistry transformations and protecting group methodologies (protection and deprotection) useful in synthesizing the compounds identified by the methods described herein are known in the art and include, for example, those such as described in R. Larock, Comprehensive Organic Transformations, VCH Publishers (1989); T. W. Greene and P. G. M. Wuts, Protective Groups in Organic Synthesis, 2nd ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Libraries of compounds may be presented in solution (e.g., Houghten, Biotechniques 13:412-421, 1992), or on beads (Lam, Nature 354:82-84, 1991), chips (Fodor, Nature 364:555-556, 1993), bacteria (Ladner, U.S. Pat. No. 5,223,409), spores (Ladner U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al. Proc. Natl. Acad. Sci. 87:6378-6382, 1990; Felici, J. Mol. Biol. 222:301-310, 1991; Ladner supra.).

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to have reprogramming or regeneration inducing activity further fractionation of the positive lead extract is necessary to isolate molecular constituents responsible for the observed effect. Thus, the goal of the extraction, fractionation, and purification process is the careful characterization and identification of a chemical entity within the crude extract that reprograms a cell (e.g., an adult cell or embryonic stem cell) or that enhances regeneration. Methods of fractionation and purification of such heterogeneous extracts are known in the art. If desired, compounds shown to be useful as therapeutics are chemically modified according to methods known in the art.

### Therapeutic Methods

The invention provides for the treatment of diseases and disorders associated with a deficiency in cell number (e.g., a reduction in the number of pancreatic cells) or an insufficient level of cell biological activity (e.g., a deficiency in insulin production). For example, the invention provides compositions for the treatment of diabetic patients who lack sufficient levels of insulin due to a decrease in the number or activity of insulin producing pancreatic cells. Many diseases associated with a deficiency in cell number are characterized by an increase in cell death. Such diseases include, but are not limited to, neurodegenerative disorders, stroke, myocardial infarction, or ischemic injury. Injuries associated with trauma can also result in a deficiency in cell number in the area sustaining the wound. Methods of the invention ameliorate such diseases, disorders, or injuries by generating cells (e.g., myocardial cells, neurons, insulin-expressing cells) that can supplement the deficiency. Such cells are generated from the reprogramming of a cell to a cell type of interest (e.g., the reprogramming of a liver cell to an insulin producing cell) or by promoting the regeneration of a cell, tissue, or organ. In general, the invention provides a method for reprogramming a cell that involves contacting the cell (e.g., adipocytes, bone marrow derived cells, epidermal cells, endothelial cells, fibroblasts, hematopoietic cells, hepatocytes, myocytes, neurons, pancreatic cells, and their progenitor cells or stem cells) with a fusion protein comprising a transcription factor polypeptide or fragment thereof fused to or containing a protein transduction domain; and altering the expression level of at least one, two, three, four, five or more polypeptides in the cell, thereby reprogramming the cell.

In one embodiment, a fusion polypeptide or polypeptide containing a protein transduction domain is administered to a cell, tissue, or organ in situ to ameliorate a deficiency in cell number. Alternatively, the polypeptide is administered to cells in vitro and then the cells containing the polypeptide (or nucleic acid molecules encoding them) are administered to the patient to ameliorate the disease, disorder, or injury. The polypeptide is delivered to those cells in a form in which it can be taken up by the cells, such that sufficient levels of protein are transduced to ameliorate a disease or disorder. In one embodiment, a therapeutic polypeptide or fusion polypeptide is delivered locally to a site where an increase in regeneration or where cellular reprogramming is desired. Administration may be my any means sufficient to result in a sufficient level of cellular transduction. While the particular level of transduction will vary depending on the therapeutic objective to be achieved, desirably at least 2, 5, 10, or 15% of the cell of a tissue are transduced. In other embodiments, at least 25%, 35%, or 50% of cells are transduced. In still other embodiments, at least 75%, 85%, 95% or more of cells are transduced. Preferably, levels of a polypeptide are altered by at least about 5%, 10%, 25%, 50%, 75% or more.

In various embodiments, fusion polypeptides are administered by local injection to a site of disease or injury, by sustained infusion, or by micro-injection under surgical conditions (Wolff et al., Science 247:1465, 1990). In other embodiments, the fusion polypeptides are administered systemically to a tissue or organ of a patient having a deficiency in cell number that can be ameliorated by cell regeneration or reprogramming.

In another approach cellular transduction into the affected tissue of a patient is accomplished by transferring a fusion polypeptide of the invention into a cultivatable cell type ex vivo (e.g., an autologous or heterologous primary cell or progeny thereof), after which the cell (or its descendants) are injected into a targeted tissue at the site of disease or injury. In some embodiments, the cells are present in a cellular matrix that provides for their survival, proliferation, or biological activity. Another therapeutic approach included in the invention involves administration of a recombinant therapeutic fusion polypeptide, biologically active fragment, or variant thereof.

The present invention provides methods of treating disease and/or disorders or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a disease or disorder or symptom thereof characterized by a deficiency in cell number. The method includes the step of administering to the mammal a therapeutic amount of an amount of a composition of the invention sufficient to treat the disease or disorder or symptom thereof, under conditions such that the disease or disorder is treated.

In other embodiments, therapeutic polypeptides of the invention are produced in a cell transduced with a viral (e.g., retroviral, adenoviral, and adeno-associated viral) vector that is used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a nucleic acid molecule, or a portion thereof, that encodes a therapeutic protein of the invention can be cloned into a retroviral vector and expression can be driven from its endogenous promoter, from the retroviral long terminal repeat, or from a promoter specific for a target cell type of interest (e.g., a cell of the central nervous system). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer the gene of interest systemically or to a cell at the site that requires cell reprogramming or an increase in regeneration.

The methods herein include administering to the subject (including a subject identified as in need of such treatment) an effective amount of a fusion polypeptide described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compositions herein may be also used in the treatment of any other disorders in which a deficiency in cell number may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with a deficiency in cell number, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Pancreatic Transcription Factor Polynucleotide Therapy

The invention further provides methods for persistently expressing pancreatic transcription factors in a cell, tissue, or organ that does not typically express such proteins. If desired, a viral vector (e.g., an adeno-associated viral vector) is used to persistently express a Pdx-1 and/or NeuroD polypeptide or fusion polypeptide (e.g., PTD-Pdx-1, PTD-NeuroD). Such viral vectors may, if desired, be administered in combination with fusion polypeptides of the invention. Advantageously, the fusion polypeptides containing a pancreatic transcription factor and a protein transduction domain are only transiently present in the cell, while the polypeptide or fusion polypeptide expressed in the adeno-associated viral vector are persistently expressed. Polynucleotide therapy featuring a polynucleotide (e.g., an AAV expression vector, such as an AAV-2 vector) encoding a Pdx-1, Pdx-1/VP16, Ngn3, Pax4, NeuroD1, Nkx2.2, Nkx6.1, Is11, Pax6, or MafA protein, variant, or fragment thereof is one therapeutic approach for treating hyperglycemia. Such nucleic acid molecules can be delivered to cells of a subject having hyperglycemia (e.g., hyperglycemia related to type 1 or type 2 diabetes). The nucleic acid molecules must be delivered to the cells of a subject in a form in which they can be taken up so that therapeutically effective levels of a pancreatic transcription factor, such as Pdx-1, Pdx-1/VP16, Ngn3, Pax4, NeuroD1, Nkx2.2, Nkx6.1, Is11, or Pax6, or fragments thereof can be produced. Preferably, persistent expression of an islet-1, Pdx1, neuroD, Nkx6.1, or MafA polypeptide (e.g., a PTD fusion polypeptide) is maintained at an effective level for longer than 1 week, 2 weeks, 3 weeks, or longer than 1, 3, 6, or 12 months. If desired, the persistent expression of a pancreatic transcription factor is combined with the transient presence of a Ngn3 and/or Pax4 polypeptide (e.g., a PTD fusion polypeptide).

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding pancreatic transcription factor protein, variant, or fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). In one embodiment, an adeno-associated viral vector (e.g., serotype 2) is used to administer a polynucleotide to the liver or to a lobe of the liver.

Non-viral approaches can also be employed for the introduction of a therapeutic to a cell of a patient requiring modulation of hyperglycemia. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). In one embodiment, the nucleic acids are administered in combination with a liposome and protamine.

Gene transfer can also be achieved using non-viral means involving transfection in vitro. Such methods include the use of calcium phosphate, DEAF dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue or delivered via a canula.

cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types (e.g., hepatocytes, hepatic stem cells, or other liver-derived cell) can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant pancreatic transcription factor protein or fusion protein, variant, or fragment thereof, either directly to the site of a potential or actual disease-affected tissue, to an organ where the polypeptide will have a therapeutic effect, or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgment of the person administering or supervising the administration of the compositions.

### Polypeptide and Polynucleotide Therapeutics

The invention provides a simple means for identifying compositions (including pancreatic transcription factors, protein transduction domain/fusion polypeptides, fragments thereof, nucleic acid molecules encoding such proteins, peptides, small molecule inhibitors, and mimetics) capable of acting as therapeutics for the treatment of a disease or disorder characterized by a deficiency in cell number or in biological activity. Accordingly, a polypeptide, such as a transcription factor, or other agent discovered to have medicinal value by reprogramming a cell to another cell type or by promoting regeneration are identified using the methods described herein. Such polypeptides are useful as a therapeutic agents or as information for the structural modification of existing polypeptides, e.g., by rational drug design. Such methods are useful for screening agents having an effect on a variety of conditions characterized by a deficiency in a cell type of interest.

For therapeutic uses, a fusion polypeptide identified using the methods disclosed herein may be administered systemically, for example, formulated in a pharmaceutically-acceptable buffer such as physiological saline. Preferable routes of administration include, for example, subcutaneous, intravenous, intraperitoneally, intramuscular, or intradermal injections that provide continuous, sustained levels of the drug in the patient. Treatment of human patients or other animals will be carried out using a therapeutically effective amount of a polypeptide or nucleic acid molecule therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the cellular deficiency. Generally, amounts will be in the range of those used for other therapeutic polypeptide or protein therapy agents used in the treatment of other diseases. In one embodiment, fusion polypeptides of the invention are administered at a dosage that controls the clinical or physiological symptoms of hyperglycemia as determined by a diagnostic method known to one skilled in the art, or using any that assay that measures the expression or the biological activity of pancreatic transcription factor polypeptide, such as the expression of a target gene. In one embodiment, compositions of the invention are administered in an effective amount of at least about 1-5 mg/Kg body weight or at least about 5 μg/g body weight, 0.1 mg/20 g body weigh, or 1 mg/20 g. In other embodiments, at least about 0.5, 1, 2, 3, 4, 5, 6, 7, 8, 9, 10, 12, 15, or 20 mg/kg of a polypeptide therapeutic of the invention is administered.

### Formulation of Pharmaceutical Compositions

The administration of a composition of the invention for the treatment of a disease, disorder, or injury characterized by a deficiency in cell number may be by any suitable means that results in a concentration of the therapeutic that, combined with other components, is effective in ameliorating, reducing, or stabilizing the disease. For example, an amount that reduces or normalizes blood glucose levels in a subject. A therapeutic polypeptide, polynucleotide, or cell comprising such agents may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). Desirably, the polypeptide may be modified or formulated to enhance polypeptide half-life, increase absorption, or provide for sustained release.

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the peritoneal cavity or at another site where distribution of the composition is desired; (v) formulations that allow for convenient dosing, such that doses are administered, for example, once every one to two days, or once every one to two weeks; and (vi) formulations that target an disease, disorder, or injury by using carriers or chemical derivatives to deliver the therapeutic agent to a particular cell type (e.g., liver cell or pancreatic cell) whose function is perturbed in a subject having the disease, disorder, or injury. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

If desired, therapeutic compositions of the invention are provided together with other agents that enhance the regeneration of a cell of interest or that enhance the reprogramming of a cell of interest. In one embodiment, the agents are growth factors, such as soluble growth factors. For example, a therapeutic polypeptide, polynucleotide, or cell comprising such agents is provided together with a soluble growth factors, such as PDGF, EGF, VEGF, bFGF, HGF, NGF, KGF) or is provided together with a beta cell promoting factor, such as nicotinamide, exentin 4, GLP-1, betacellulin, Islet neogenesis associated protein (INGAP), or Ghrelin.

### Methods of Delivery

The pharmaceutical composition may be administered by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. In one embodiment, a therapeutic composition of the invention is provided via an osmotic pump. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation or it may be presented as a dry powder to be reconstituted with water or another suitable vehicle before use. Apart from the active polypeptide therapeutic(s), the composition may include suitable parenterally acceptable carriers and/or excipients. The active polypeptide therapeutic (s) may be incorporated into an osmotic pump, microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active fusion polypeptide therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

In one embodiment, a therapeutic composition of the invention (e.g., polypeptide, polynucleotide, or cell comprising such agents) is provided locally via a canula. For example, for reprogramming a liver-derived cell to insulin producing cell a composition of the invention is provided to the liver via the portal vein. More preferably, the composition is directed specifically to a single lobe of the liver by providing the composition (e.g., via a canula) to only one of the three branches of the portal vein, such that only one lobe of the liver comprises insulin producing cells. In other embodiments, a composition of the invention is provided via an osmotic pump. Desirably, the osmotic pump provides for the controlled release of the composition over 1-3 days, 3-5 days, 5-7 days, or for 2, 3, 4, or 5 weeks.

### Combination Therapies

Compositions of the invention may, if desired, be delivered in combination with any other polypeptide or polynucleotide therapeutic of the invention, including detectably labelled fusion proteins to assist in monitoring the efficacy of the protein therapy, or with any conventional therapeutic known in the art. In one embodiment, a fusion polypeptide of the invention, such as a pancreatic transcription factor fused to a protein transduction domain, is used to reduce hyperglycemia in a diabetic subject. This therapeutic effect is desirable even if the therapeutic method does not entirely eliminate the patient's dependence on insulin. Accordingly, fusion polypeptides of the invention may be administered together with insulin to alleviate hyperglycemia or a symptom or complication thereof. Desirably, a therapeutic fusion polypeptide of the invention reduces a patient's dependence on insulin by at least about 5, 10, or 15%, more desirably by at least about 20%, 25%, or even by 30%, or even more desirably by 50%, 75%, 85% or more. In other embodiments, the polypeptide therapeutic is combined with a polynucleotide of the invention (e.g., a polynucleotide encoding a pancreatic transcription factor or fusion protein). In other embodiments, compositions of the invention are used in combination with diet, weight loss, or oral, injectable, nasal or other insulin therapies to reduce and/or normalize blood glucose levels. Combinations of the invention may be formulated together and administered simultaneously or may be administered within twenty-four hours, within 2, 3, or 5 days, or within 1, 2, 3 or 5 weeks of each other.

### Kits or Pharmaceutical Systems

The present compositions may be assembled into kits or pharmaceutical systems for use in ameliorating hyperglycemia. Kits or pharmaceutical systems according to this aspect of the invention comprise a carrier means, such as a box, carton, tube or the like, having in close confinement therein one or more container means, such as vials, tubes, ampoules, bottles and the like. The kits or pharmaceutical systems of the invention may also comprise associated instructions for using the agents of the invention.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

### EXAMPLES

The studies described herein provide for the therapeutic and or prophylactic use of protein transduction domain fusion proteins, including protein transduction domain (PTD) pancreatic transcription factor (PTF) fusion proteins. In one embodiment, PTD-PTF fusion proteins are used to direct the reprogramming or transdifferentiation of liver cells into pancreatic endocrine precursor cells or other insulin producing cells. The PTD-PTFs of the invention are used to reverse hyperglycemia in diabetic subjects. In brief, the invention provides constructs encoding a PTF fusion genes (Pdx1, Pdx1-VP16, Ngn3, and Pax4) with or without an 11 amino acid TAT protein transduction domain (PTD). These constructs have been used to produce and purify PTD-Ngn3, Ngn3, and Pax4 fusion proteins that are biologically active in vivo. The invention provides compositions and methods for monitoring the transdifferentiation process, including color-coded reporter PTF genes that are used to analyze transdifferentiation events occurring at cellular and molecular levels. The feasibility of reprogramming the liver cells into liver-derived pancreatic precursors that subsequently mature into functional pancreatic beta-cell-like insulin producing cells following cell transplantation, and restore normoglycemia in diabetic mice is supported by the viral-mediated expression of pancreatic transcription factor-transgenes expression in diabetic mice. Advantageously, Ngn3 and Pax4 fusion proteins of the invention are only transiently present in the cells described herein. This transient presence is sufficient to generate liver-derived glucose-regulated, fully functional insulin producing cells.

Studies described herein suggest that the present invention provides certain advantages over methods that provide for the sustained expression of Ngn3 and Pax4. In particular, persistent lentiviral-mediated Ngn3 expression caused hepatic cells to exit the cell cycle and undergo apoptosis, while persistent LV-mediated Pax4 expression in Pdx1-VP16-expressing IPCs resulted in severe hypoglycemia and the death of the implanted mice.

### Example 1

### Protein Transduction Domain-Containing-Ngn3 Fusion Protein (PTD-Ngn3)

A schematic showing the structure of exemplary protein transduction domain (PTD) containing fusion proteins is provided at FIG. 1B. While the orientation of the various domains is shown in one particular configuration, this is merely provided as one example. Other combinations and configurations are within the scope of the invention. In particular, the PTD domain may be positioned at the carboxy or amino terminal or at any other position within the polypeptide.

A PTD-Ngn3-V5-His-tag or Ngn3-V5-His-tag fusion protein was generated using pCR T7/CT-TOPO expression plasmid (Invitrogen). Because the anti-His-tag antibody also recognizes many histidine-rich proteins, the pCR T7/CT-TOPO plasmid also codes for V5 epitope, offering the advantage of selective detection of recombinant proteins using high quality, commercially available anti-V5 antibody. In brief, the cDNA encoding the entire reading frame of mouse Ngn3 was used to generate PCR products with primers containing the PTD (YGRKKRRQRRR)-sequence. (All PCR products were confirmed by sequencing in the University of Florida DNA Core Facility.) The PTD-Ngn3 and Ngn3 PCR products were cloned into an expression plasmid, pCR T7/CT-TOPO plasmid (Invitrogen), allowing the generation of V5 and his-tagged fusion proteins. The resulting vectors are designated pCR-PTD-Ngn3-V5-His or pCR-Ngn3-V5-His (PTD-minus control). The final cDNA encodes peptide sequences in the following order: PTD (11aa)-Ngn3 (214aa)-V5 epitope (14aa)-His-tag (6aa). The resulting fusion protein retained Ngn3's biological function.

While specific protein transduction domain (PTD)-containing fusion proteins are described, one skilled in the art appreciates that the invention is not so limited. The position of each domain can be varied to enhance, for example, the biological activity, transduction, solubility, or expression of the protein in a host cell. For example, the PTD domain may be present at the carboxy or amino terminal of the polypeptide or may be located at a position anywhere within the molecule.

### Example 2

### Production and Purification of PTD-Ngn3-V5-His Fusion Protein

To produce the desired fusion proteins, Escherichia coli BL21(DE3) cells transformed with plasmids pCR-PTD-Ngn3-V5-His or pCR-Ngn3-V5-His were grown at a 37° C. in LB medium containing ampicillin (100 μg/ml) to an OD₆₀₀ 0.5 (in mid-log phase). Expression of the fusion proteins was induced by adding 0.5 mM isopropylthiogalactoside (IPTG) for 4 hours. The induced cells were harvested and lysed by sonication in Lysis buffer (Invitrogen). Soluble PTD-Ngn3 or Ngn3 fusion proteins were purified using a Ni-NTA agarose column (Invitrogen), followed by desalting with a PD10 column (Amersham) according to the manufacturer's instructions. The purified proteins were visualized by Coomassie blue staining (FIGS. 2A and 2C) and confirmed by Western blot analysis with anti-V5 antibody (FIG. 2B). The proteins were aliquoted in PBS with 10% glycerol and stored at −80° C. until use. Purified PTD-Ngn3 and Ngn3 fusion proteins (FIG. 2C) show slight difference in molecular weight (FIG. 2C).

### Example 3

### Functional Characterization of PTD Domain-Containing Ngn3 Fusion Protein

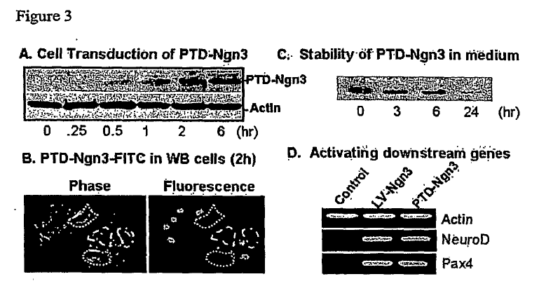

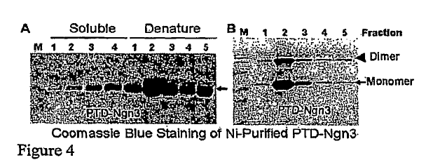

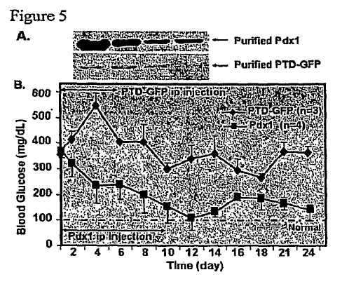

To evaluate ability of the PTD fusion protein to penetrate cells, a time-course of PTD-Ngn3 transduction was performed. WB cells, which are a rat hepatic epithelial stem-like clonal cell line (Tsao et al., Exp. Cell Res., 154, 38-52, 1984), were incubated with medium containing purified PTD-Ngn3 fusion protein (0.2 μM) for various periods, washed three times with PBS, and harvested in 2× SDS sample buffer. The PTD-Ngn3-V5 fusion protein was detected by Western blot with anti-V5 antibody (FIG. 3A). The results of this analysis demonstrated that protein transduction mediated by the PTD domain peaked at two hours. To visualize this process, purified PTD-Ngn3 fusion protein was labeled with FITC according to the manufacturer's instruction (Pierce). PTD-Ngn3-V5-*FITC was added to WB cells at a final concentration of 0.2 μM for 2 hours. The Ngn3 fusion protein lacking the PTD failed to enter cells. FIG. 3B shows that >70% of the cells contained FITC-labeled fusion protein both in the cytoplasm and nuclei. To determine the stability of the PTD-Ngn3 fusion protein in cell culture medium, the fusion protein (0.2 μM) was added to WB cells and aliquoted medium was removed at indicated times. The fusion protein was detected by Western blot with anti-VS antibody (FIG. 3C). At twenty-four hours the PTD-Ngn3 protein is still present in the culture medium, although there was a decrease in the protein level. FIGS. 4A and 4B show the quality of the purified soluble and insoluble PTD-Ngn3 fusion protein.

To determine if the PTD-Ngn3 can activate NeuroD and Pax4, which are downstream target genes, human Huh7 cells were treated with either LV-Ngn3 (MOI=20) or PTD-Ngn3 (0.2 μM, fresh PTD-Ngn3 was added every six hours) for four days. RNA was collected and RT-PCR was performed. FIG. 3D shows equivalent potency in Ngn3 activation of NeuroD and Pax4 (down stream target genes) between LV-Ngn3 and PTD-Ngn3 protein treatments. These results indicate that biologically active PTD-Ngn3 fusion protein was produced and that this protein was detectable by the anti-V5 antibody.

### Example 4

### PTD-Pdx1-VP16 (PTD-PV) or PTD-Pdx1-VP16/PTD-Ngn3 Intraperitoneal Injection Restored Blood Glucose Levels of Diabetic Mice

Pdx1 acts as a pancreatic “master” control gene. Streptozotocin-induced diabetic mice were injected daily with either his-tag purified soluble PTD-Pdx1 fusion protein or PTD-GFP fusion protein (100 μg/mouse) for 10 days and the blood glucose levels were monitored every other day by tapping the tail vein. FIGS. 5A and 5B show the results of these experiments. FIG. 5A shows purified proteins used for injections; and FIG. 5B show that multiple intraperitoneal injections of soluble Pdx1 protein reversed hyperglycemia to nearly normal glycemia. No effect on blood glucose levels was observed in control mice receiving PTD-GFP injections.

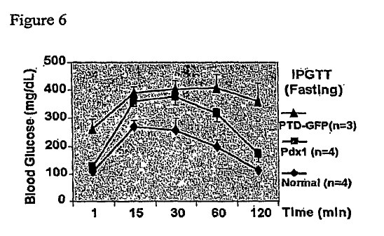

Intraperitoneal glucose tolerant tests (IPGTT) were performed on normal, PTD-GFP-, and PTD-Pdx1 fusion protein injected mice (FIG. 6). Mice injected with the PTD-Pdx1 fusion protein showed a virtually normal IPGTT curve (squares) relative to normal mice (diamonds). In contrast, mice receiving PTD-GFP were not able to reduce the extra dosage of glucose. These results indicated that intraperitoneal injection of soluble PTD Pdx1 fusion protein reduced blood glucose levels in diabetic mice and that these results were statistically significant. Without wishing to be bound by theory, it is possible that the PTD Pdx1 fusion protein entered pancreatic cells and promoted pancreatic beta cell regeneration; alternatively, the PTD Pdx1 fusion protein may have promoted liver cell transdifferentiation into insulin promoting cells following entry into the liver cells via a portal vein delivery system after the protein was translocated into mesenteric vascular systems (capillary, small veins, and lymphatic channel).

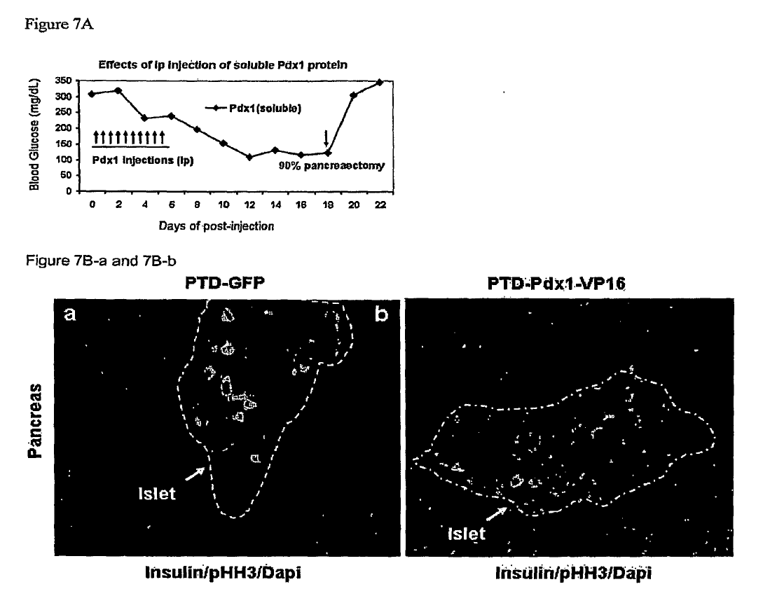

To determine that whether pancreatic beta cell regeneration played a role in restoring normoglycemia, pancreactomy was performed in a mouse receiving soluble PTD Pdx1 fusion protein injection. This operation removed >90% of the pancreas. Blood glucose levels were monitored after the operation. The mouse was provided with insulin daily injections (PID) and daily soluble Pdx1 fusion proteins for five days at a 100 μg dosage. The blood glucose levels were monitored. FIG. 7A shows that a rapid rebound from hyperglycemia is observed in this mouse. Normoglycemia was achieved by repeated intraperitoneal PTD Pdx1 fusion protein injections. These results were surprising given that 90% removal of the pancreas by pancreactomy typically results in persistent hyperglycemia. FIG. 7B indicates that Pdx1 or PTD-Pdx1-VP16 intraperitoneal injection promotes pancreatic islet cell regeneration. Histone H3, a protein involved in chromatin structure, is specifically phosphorylated (at serine 10) during chromatin condensation in mitosis. An antibody to phospho-histone H3 (pHH3) was used as a reliable method to detect mitotic activity in cells.

In another experiment, two mice with streptozotocin-induced diabetes were injected intraperitoneally daily for five days with purified soluble fusion proteins of PTD-PV or PTD-PV/PTD-Ngn3 (50 μg/mouse). Pdx1-VP16 is an activated form of Pdx1 (Pdx1-VP16). The mice blood glucose levels were monitored every two days. The mice were sacrificed at day twenty-seven post-injection.

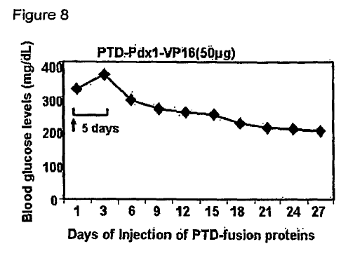

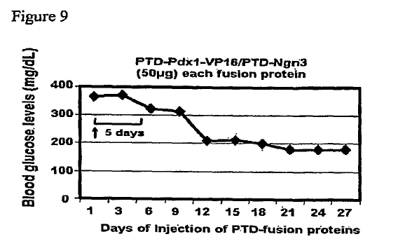

FIGS. 8 and 9 show changes in the mouse blood glucose levels. As shown in FIG. 8, intraperitoneal injection of soluble PTD-Pdx1-VP16 reduced blood glucose levels from ˜400 mg/dl to ˜200 mg/dl less than three weeks post-injection of the diabetic mouse. Furthermore, a combination of PTD-PV/PTD-Ngn3 showed a synergistic effect on reducing blood glucose levels from ˜385 mg/dl to 200 mg/dl within two weeks (FIG. 9). The body weights of both mice were stable.

### Example 5

### Pancreatic Beta Cell Regeneration

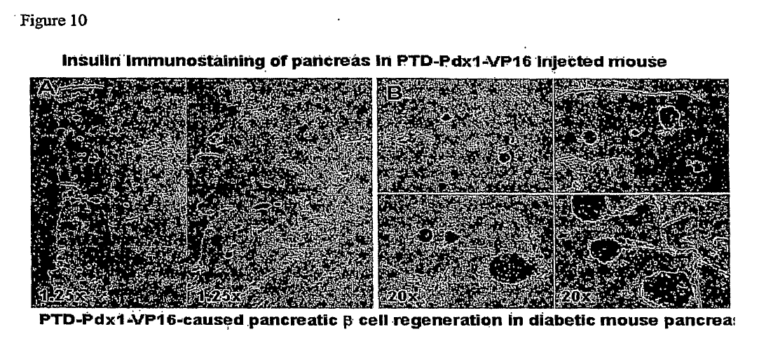

To examine the mechanism responsible for the observed reduction in blood glucose levels, the mice were sacrificed and the pancreas, liver, spleen, kidneys, heart, and lungs were fixed in 10% formalin for histological examination. FIGS. 10A and 10B show the result of insulin immunostaining on the PTD-Pdx1-VP16-injected mouse pancreas. These results indicate that there is robust pancreatic beta-cell regeneration in the pancreas of these injected mice. Similar results were seen in PTD-PV/PTD-Ngn3-injected mouse pancreas. FIG. 10A shows a representative section of the pancreas that includes the entire pancreatic islet beta cells. FIG. 10B shows that the newly regenerated islets vary in sizes with many small islets. Most newly regenerated islets are close to or adjacent to pancreatic ducts, suggesting the presence of beta cell neogenesis. Some scattered singly insulin-positive beta cells were adjacent to the exocrine glands suggesting exocrine cell transdifferentiation into endocrine beta cells. One mechanism for pancreatic beta cell regeneration is residual beta cell replication.

### Example 6

### Promotion of Liver Cell Transdifferentiation into Pancreatic Endocrine Cells Including Insulin-Producing Cells

The liver was sectioned and immunostained with an anti-insulin antibody. FIGS. 11A-11D show scattered insulin-positive cells noted in the liver section (FIG. 11B) of the mouse who received PTD-Pdx1-VP16 soluble protein injection. No insulin-positive cells were detected in the liver section (D) of the mouse receiving PTD-GFP fusion protein. Scattered insulin-positive cells were also detected by anti-insulin antibody in the liver sections (FIG. 12) of the mouse who received injected PTD-Pdx1-VP16/PTD-Ngn3 fusion proteins. These results indicate that the reduction of the mouse blood glucose levels are attributable to pancreatic beta cell regeneration and liver-cell-to-endocrine pancreas transdifferentiation mediated by PTD-PTF (Pdx1-VP16, or Pdx1-VP16/Ngn3).

These results indicate that multiple intraperitoneal injections of modified pancreatic “master” control gene, soluble PTD-Pdx1-VP16 protein or a combination of soluble PTD-Pdx1-VP16 and PTD-Ngn3 proteins into streptozotocin-induced diabetic mice had a synergistic effect in reducing blood glucose levels and ameliorated diabetes in mice. This reduction in mouse blood glucose levels was due to an increase in the number of insulin producing cells resulting from endogenous pancreatic beta-cell regeneration and liver cell transdifferentiation. These results demonstrated that delivery of pancreatic transcription factors via a protein transduction domain (PTD) can be used successfully in vivo to induce pancreatic beta cell and to reprogram hepatocytes into insulin-producing cells.

The successful use of this approach to treat diabetes in mice, indicates that a combination of pancreatic transcription factors (Pdx1-VP16, and Ngn3) delivered using PTD technology is useful for the amelioration of both type 1 and type 2 diabetes in humans. The treatment can be implemented by injecting recombinant human PTD-Pdx1-VP16 and PTD-Ngn3 proteins into the abdominal cavity or portal vein to promote endogenous pancreatic beta cell regeneration and/or liver cell transdifferentiation, which in turn, will treat diabetes and prevent metabolic complications associated with persistent hyperglycemia.

### Example 7

### Portal Vein Injection of AAV-PTFs (Pdx1-VP16, Ngn3, or Both) Reduced Blood Glucose Levels in Diabetic Mice

The feasibility of in vitro reprogramming liver stem cells into functional insulin-producing β-cell surrogates by introducing genes for key pancreatic transcription factors (i.e. Pdx1, Pdx1-VP16, Ngn3, and Pax4) is demonstrated herein. In addition, the present results indicate effective transcription factor combinations (Pdx1-VP16 combined with Pax4 and/or Pdx1-VP16 with Ngn3) to be used in reprogramming. In particular embodiments, the invention provides viral vectors expressing a pancreatic transcription factor that can be used for the persistent expression of a pancreatic transcription factor of interest. Such viral vectors can be used in combination with fusion polypeptides containing a pancreatic transcription factor that is only transiently present in a cell.

Three groups of diabetic mice were injected with AAV-Pdx1-VP16, AAV-Ngn3, or AAV-Pdx1-VP16/AAV-Ngn3 at 1×10⁸ viral particles via the portal vein with one group of mice receiving AAV-GFP serving as control for AAV virus. The blood glucose levels were monitored every two days and the glucose tolerance test was performed on mice with nearly normalized blood glucose levels. One mouse from each group was killed and the liver and pancreas were examined for presence of beta cell regeneration and pancreatic endocrine cells in the liver. FIGS. 13A shows that the liver cell transduction efficiency of AAV-serotype 2 is approximately 20% overall. FIG. 13B shows that a combination of AAV-Pdx1-VP16 with AAV-Ngn3 effectively reduced blood glucose levels in diabetic mice and restored the mouse ability of responding a high glucose challenge. AAV-Ngn3 alone shows no significant effect on blood glucose reduction. AAV-Pdx1-VP16 shows moderate effect on reducing the blood glucose levels.

### Example 8

### Liver-Derived Insulin-Positive Cells

Paraffin sections of the liver from mice that received AAV-PV, AAV-Ngn3, or AAV-PV/AAV-Ngn3 virus were examined by immunostaining with anti-insulin and glucagon antibodies. FIG. 14 (left panel) shows that AAV-PV portal vein injection converted some hepatocytes into strong insulin-positive cells. This result is consistent with the reduction in blood glucose levels identified in these animals (FIG. 13B).

Liver sections of animals that received AAV-PV/AAV-Ngn3 via portal injection show many insulin-positive cells. Most of these cells are distributed around the central veins (FIG. 15). Most insulin-positive cells were located on the periphery under the liver capsule. Consistent with this expression, blood glucose levels were normalized in mice that received AAV-PV/AAV-Ngn3 via portal injection. Although the distribution of the insulin-positive cells varies from field to field, the overall percentage of insulin-positive cells is estimated to be 2-3% of the total number of hepatocytes. No significant reduction of the blood glucose levels was observed in the mice that received AAV-Ngn3 injection alone. Only scattered insulin-positive liver cells were identified in liver sections from these mice (FIG. 14, right panel).

### Example 9

### Liver-Derived Glucagon Positive Cells

To determine whether hepatocytes can be converted into pancreatic endocrine cells by pancreatic transcription factors (PTFs), paraffin sections of the liver from the mice that received AAV-PV, AAV-Ngn3, or AAV-PV/AAV-Ngn3 virus were immunostained with an anti-glucagon antibody. These studies identified glucagon-positive hepatocytes in liver. Most glucagon-positive hepatocytes were located next to central veins. FIG. 16 shows the results of a liver section of glucagon immunostaining from a mouse that received AAV-PV/AAV-Ngn3 portal vein injection.

A combination of recombinant adeno-associated viruses (AAV) encoding Pdx1-VP16 and Ngn3 were administered via the portal vein to mice with streptozotocin-induced diabetes. Mice that received this combination showed a synergistic reduction in blood glucose levels, indicating that the expression of Pdx1-VP16 and Ngn3 ameliorated diabetes in these mice. This effect was mediated in large part by liver cell transdifferentiation into insulin-producing cells and to a lesser degree by endogenous pancreatic beta-cell regeneration. Portal vein injection of AAV-Pdx1-VP16 alone significantly reduced blood glucose levels. This effect is consistent with histological results, which showed the presence of insulin- and glucagon-positive cells in liver sections. To determine whether these effects were due in part to pancreas regeneration, the pancreases of these mice were assayed for insulin immunostaining. Lower levels of pancreatic beta cell regeneration were identified in mice that received AAV-PTF via portal vein injection than were observed in mice that received the vectors by interperitoneal injection. Portal vein injection of AAV-Ngn3 alone showed no significant effect on blood glucose levels. Only scattered insulin- and glucagon-positive cells were identified in liver sections from these mice.

Given that key pancreatic genes, such as Pdx1-VP16 and Ngn3, can be delivered via the portal vein to successfully transdifferentiate hepatocytes in the liver into insulin-producing cells, it is likely that pancreatic transcription factors fused to protein transduction domains could be delivered to the liver via the portal vein and that these fusion proteins could be used to transdifferentiate hepatocytes in vivo as well as to promote beta cell regeneration. Such methods are useful for the treatment of patients with both type 1 and type 2 diabetes and for the prevention of complications associated with persistent hyperglycemia.

### Example 10

### Sequential Delivery of Pancreatic Transcription Factor Fusion Protein Therapy

Protein therapy is provided for in vivo regeneration and reprogramming using three pancreatic transcription factors which are sequentially expressed during beta-cell differentiation as shown in FIG. 17, which provides a summary of the sequential order and duration of expression of the particular PTFs (Soria Differentiation 2001; 68(4-5):205-219; Hui et al., Eur J Endocrinol 2002; 146(2):129-141). Sequences of exemplary pancreatic transcription factors are provided at FIGS. 18A-18I. Pdx1 is expressed in the developing pancreas in a biphasic fashion. It first peaks between embryonic day (E) 9.5 to E10 (expressed in all pancreatic precursor cells) and expression lasts about 2-3 days. A low-level expression of Pdx1 then follows a 5-6 day time interval. During this interval, Ngn3 expression begins at E11, peaks at E15, and lasts for 2-4 days. Ngn3 expression determines cell differentiation towards a pancreatic endocrine cell fate. Pax4 appears as Ngn3 is disappearing and lasts ˜1-3 days. Pax4 is expressed only in developing beta cells. Following Pax4 activation, Pdx1 reappears, is permanently expressed in the differentiated beta-cells, and maintains beta-cell function. FIG. 17B illustrates the administration of PTD-PTF fusion proteins that mimics the natural course of the PTF expression sequence illustrated in FIG. 17A. Methods for delivering pancreatic transcription factor fusion polypeptides mirror the in vivo expression of such factors during development.

In particular, the tissue kinetics and tissue distribution of the PTD-GFP fusion proteins is monitored in mice. 100 μg (˜5 μg/g body weight) of PTD-GFP fusion protein is administrated to mice through three routes (portal vein, i.v., and i.p.) and tissues are harvested at 1, 2, 5, 10, and 24 hours after treatment. The mean half-life for PTD-GFP or PTD-PTFs is estimated by Western blot and quantified by densitometry (Cai et al., Eur J Pharm Sci 2005; Kaneto et al., Nat Med 2004; 10(10):1128-1132). The distribution of PTD-PTF proteins in various tissues is examined based on the PTD-GFP data. PTD-PTF proteins in various tissue is analyzed by immunohistochemical staining, as described by Xiong et al., Stem Cells Dev 2005; 14(4):367-377; and by Western blot analysis with anti-V5 antibodies as described in the preliminary studies. Based on the results of these studies, PTD-PTF fusion proteins intraperitoneally delivered or portal vein injection delivery methods to streptozotocin-induced diabetic mice are optimized to mirror the endogenous expression pattern of these factors during development. Subsequently, the effect on liver reprogramming or pancreas regeneration is monitored as described above.

### Example 11

### Generation of Reporter Gene Constructs

Plasmids containing promoter genes coupled with fluorescent color-coded protein including rat insulin-1 promoter eGFP, NeuroD-eGFP, Nkx2.2-RFP, and Pax4-RFP were generated in lentiviral vectors. These fluorescent color coded gene reporter are used for monitoring stage of transdifferentiation. Plasmids containing a full-length human Pax4 or mouse Nkx2.2 promoter DNA sequence were used to construct reporter fusion genes. The mouse Pax4 promoter the mouse Pax4 promoter (−2153−+1) and Nkx2.2(−1840 bp˜+21) were cloned by PCR with suitable primers into pCR 2.1-TOPO cloning vector between the restriction enzymes Xho1/Sal I (Pax4) and BamH I/Xho I (Nkx2.2). The resulting plasmids and pDsRed-Express-N1 plasmid were cut with the same restriction enzymes. The inserts of Pax4 (or Nkx2.2) promoters were purified and ligated into MCS sites of pDsRed-Express-N1 plasmid. After screening positive clones containing Pax4 or Nkx2.2 insert, the integrity of these clones were confirmed by restriction digestion and sequence analysis, and the reporter plasmids pDsRed-Pax4-RFP and pDsRed-Nkx2.2-RFP were expanded and used for in vitro transfection to test their function.

A reporter construct comprising 950 bp (−940 to +10) of huₘan NeuroD/Beta2 promoter (Miyachi et al., Brain Res Mol Brain Res 1999; 69(2):223-231) was generated by PCR with Pfu DNA polymerase (Washiobio, China) with appropriate primers using genomic DNA from human liver as template. The amplified PCR product was subjected to electrophoresis, gel purified, and cloned into pCR2.1-TOPO vector (Invitrogen) using a TA cloning kit (Invitrogen). The promoter fragment was cleaved from the plasmid by restriction digestion with EcoR1 and BamH1 and inserted into an eGFP expression vector (pEGFP-1, Clontech). The integrity of this clone was confirmed by restriction digestion and sequence analysis.

### Example 12

### Generation & Characterization of Mouse rPdx1

The antennapedia-like domain in the Pdx1 transcription factor was recently found to possess a built-in PTD, permitting this protein to bind to and penetrate cellular membranes (Noguchi et al., Diabetes 52, 1732-1737 (2003)) and to exert transcriptional function. Expression plasmids containing mouse PDX1 or PTD-GFP cDNA were first constructed with each also containing an additional nucleotide sequence coding for a hexahistidine tag at C-terminal for rapid purification using Ni²⁺-nitrilotriacetate columns. To obtain nearly homogeneous proteins in sufficient amounts for in vitro and in vivo animal studies, bacterial expression conditions were first optimized to yield 10 mg rPdx1 or PTD-GFP per liter of growth medium, and the purification protocol was improved to assure high yield and purity. FIG. 19A illustrates the structural organization of rPdx1 and PTD-GFP (top panel), and shows a Coomassie Blue-stained SDS gel indicating the purity of rPdx1 and PTD-GFP fusion proteins (left panel). These recombinant proteins consistently had a purity of at least 90-95%, based on gel densitometry. The identity of rPdx1 and GFP proteins was also confirmed by Western blotting (right panel) with anti-Pdx1 or anti-GFP antibody.

To confirm that rPdx1 protein possessed the ability to penetrate cells, WB cells were incubated with rPdx1 protein (0.2 μM final concentration) for various times, after which the cells were washed three times with PBS. Cell lysates were then collected in lysis buffer, and their proteins were separated by SDS-PAGE and blotted with anti-Pdx1 and anti-actin (control) antibodies. The relative amount of rPdx1 in the cell blots was quantified and normalized with actin by densitometry. As shown in FIG. 19B, rPdx1 protein entry commenced within five minutes. As Pdx1 incorporation proceeded, cellular rPdx1 protein level reached its peak values at 1-2 hours, and started to fall by six hours.

To determine whether the internalized rPdx1 protein was biologically active, the transcriptional function of the rPdx1 protein was assayed. WB cells were first transduced with lentivirus-containing pNeuroD-GFP reporter gene, a direct downstream target gene of the Pdx1 transcription factor. The cells were then incubated in the presence or absence of the rPdx1 protein (1 μM) for 72 hours. These cells were harvested for flow cytometry to evaluate the rPdx1-mediated NeuroD gene activity by detecting the percentage of WB cells expressing pNeuroD-GFP. To compare the efficiency of externally administered rPdx1 with cell-made Pdx1, WB cells containing a NeuroD-GFP promoter construct were transduced with lentivirus-Pdx1 vector for 72 hours and Pdx1 levels were recorded. pNeuroD-GFP-expressing WB cells served as a positive control of native cellular Pdx1 protein. Importantly, the LV transduction efficiency with WB cells approaches nearly 100%, as judged by expression of the LV-CMV-GFP vector as reported (Tang et al., Lab Invest 86, 829-841 (2006)). FIG. 19C (Left panel) shows representative fluorescence micrographs of the NeuroD-GFP-expressing cells collected on cytospin slides. Flow cytometry revealed a comparable transcription efficacy for LV-Pdx1-treated cells (21%) and rPdx1 protein-treated (19%) cells following 72-hr treatments. Both treatments showed statistically significant differences when compared to control cells containing pNeuroD-GFP vector only (FIG. 19C, right panel). These results clearly demonstrated that the rPdx1 protein is capable of rapid entry into the cells and effectively activating its downstream NeuroD gene target. These findings confirm that the rPdx1 protein has the same or comparable transcriptional activity as native Pdx1 protein produced within the cells by means of LV-Pdx1 transgene expression.

### Example 13

### In Vivo Kinetics and Tissue Distribution

While the antennapedia-like PTD allows rPdx1 protein to enter cells, relatively little is known regarding the in vivo tissue distribution of rPdx1. Therefore, to explore a safe and clinically feasible treatment for diabetes, in vivo tissue distribution and pharmacokinetics of rPdx1 protein was examined. Balb/c mice were intraperitoneally injected with 0.1 mg or 1 mg rPdx1 fusion protein per mouse. Blood samples were collected at 15 minutes, 30 minutes, 1 hour, 2 hour, 6 hour, and 24 hour, and the rPdx1 protein was detected in the sera by immunoblotting using anti-Pdx1 antibody. As shown in FIG. 20A, the appearance of rPdx1 protein in blood became evident as early as 1-hour post-injection, reaching peak values at 2 hours, and then falling markedly by 6 hours. No rPdx1 protein was detectable in the 24-hour blood sample.

For in vivo tissue distribution studies, liver, pancreas, and kidney were harvested 1 hour or 24 hour after i.p. injection and fixed in 10% formalin. Paraffin sections were immunostained with anti-Pdx1 antibody. FIG. 19B shows representative liver, pancreas, and kidney tissue images selected from animals receiving 0.1 mg rPdx1 protein at 1 hour (top-two-rows) or 24 hours (bottom row) post-injection. The Pdx1 protein was found to be concentrated in the nuclei of hepatocytes, and the Pdx1-positive cells were at greatest concentration nearest the central vein. This distribution pattern was consistent with the predicted pathway for a rapidly internalized PTD-containing protein, in this case Pdx1, entering the portal vein system via the terminal veins or capillaries. Indeed, highest Pdx1 incorporation would be anticipated for those cells nearest the central vein. Pdx1 protein was also detected in peripheral exocrine cells in the pancreas, possibly as a result of direct uptake, and along pancreatic terminal capillaries. In kidney samples, Pdx1 protein appeared in a manner consistent with the anatomic pathway for the protein filtration and elimination: Pdx1 protein was found initially in glomeruli, caps, proximal and distal tubular cells and finally accumulating in collecting duct cells. At 24 hour post-injection, only faint Pdx1 protein immunostaining was observed in liver, pancreas, and kidney sections (bottom row). Additionally, low levels of Pdx1 protein with no distinctive pattern were also detected in the tissues of the spleen, heart, lung, and brain at 1 hour post-injection, probably due to the high levels of Pdx1 in the blood and it became undetectable level at 24 hour post-injection. On the basis of these findings and data in the literature (Matsui et al. Curr. Protein Pept. Sci. 4, 151-157 (2003); Schwarze et al., Science 285, 1569-1572 (1999)), the 0.1 mg Pdx1 was chosen as the initial dosage, with a 24 hour-interval as our treatment schedule for determining the in vivo effect of the rPdx1 on diabetic mice.

### Example 14

### In Vivo Effects of rPdx1 Protein on Blood Glucose Levels in Diabetic Mice

Scheme 1 (FIG. 19C) describes the experimental strategy employed to assess the in vivo therapeutic effects of administered rPdx1 protein in Stz-induced diabetic (fasting glucose at ˜300 mg/dL) mice (Cao et al., Diabetes 53, 3168-3178 (2004); Tang et al., Lab Invest 86, 83-93 (2006)). These animals were i.p. injected with the purified rPdx1 or PTD-GFP proteins (0.1 mg or ˜5 μg/g body weight) for 10 consecutive days. PTD-containing green fluorescent protein, itself a non-therapeutic protein with an engineered PTD, served as a negative control. The fasting blood glucose levels were monitored at different time-points. As shown in FIG. 21A (bottom dark line), mice receiving rPdx1 injections achieved near normoglycemia within two weeks after first injection; however, no amelioration of hyperglycemias was observed in mice receiving PTD-GFP protein (gray line in FIG. 21B). Mice were sacrificed 14 days after the first day of protein injection, and various organ tissues were collected for tissue insulin measurements, gene expression analysis, and/or immunohistochemical studies.

At day-14 and day-40 post-injection, intraperitoneal glucose tolerance test (IPGTT) (FIG. 20B) showed that the mice receiving rPdx1 injection exhibited a nearly normal IPGTT curve (gray middle line) as that seen in normal mice (black line) at both two occasions. In contrast, those mice receiving PTD-GFP gave no indication of any ability to reduce the bolus dose of glucose (top gray line). To assess the ability of glucose-stimulated insulin release in the rPdx1-treated diabetic mice after their blood glucose levels were nearly normalized around day-14 post rPdx1 injection, normal and treated mice were challenged with i.p. bolus glucose (1 mg/g) injection and blood sera were collected at 15-min post-injection for insulin measurement. FIGS. 20C and 20D show the results of blood glucose levels (FIG. 20C) and serum insulin levels (FIG. 20D) in normal, experimental, and control mice. Sequences of oligonucleotide primers used are shown in Table 1 (below)

TABLE 1
Real time PCR pPrimer name, sequences, size, GenBank #, and PCR condition

| | | | PCR | GenBank | Tm | Cycle |
|---|---|---|---|---|---|---|
| Genes | Forward primer | Reverse primer | size | Acc. No. | (° C.) | No. |
| Actin | ACCACACCTTCTACAATGAGC | GGTACGACCAGAGGCATACA | 185 | NM_007393 | 58 | 38 |
| insulin | GCCCTTAGTGACCAGCTAT | GGA CCA CAA AGA TGC TGT TT | 167 | NM_008386.2 | 56 | 38 |
| Pdx1 | ATGAAATCCACCAAAGCTCAC | AGTTCAACATCACTGCCAGCT | 190 | NM_008814.2 | 56 | 38 |
| INGAP | GCTCTTATCTCAGGTTCAAGG | AGATACGAGGTGTCCTCCAGG | 178 | NM_013893.1 | 56 | 38 |
| Reg3g | CATGACCCGACACTGGGCTATG | GCAGACATAGGGTAACTCTAAG | 190 | NM_011260.1 | 56 | 38 |
| PAP | AATACACTTGGATTGGGCTCC | CCTCACATGTCATATCTCTCC | 195 | NM_011036.1 | 56 | 38 |

As shown in FIGS. 21C and 21D, the serum insulin levels in mice treated with rPdx1 or PTD-GFP protein were corroborated with the blood glucose levels of samples obtained on day-14 and day-40 post-injection. Glucose-stimulated insulin release for Pdx1-treated mice at 15 minutes in the IPGTT was 6.9 and 11.3 times higher than the GFP control group at day-14 and day-40, respectively, indicating a significant improvement in the ability of the rPdx1-treated mice to handle bolus dose of glucose challenge. Although the blood glucose levels at day-40 for Pdx1-treated mice is close to normoglycemia, the released insulin (2.6 μg/L) following 15 minutes of glucose-stimulation was still much lower than that in normal non-diabetic mice (5.7 μL), suggesting an immaturity of newly formed insulin-producing cells or less than optimal levels of β-cell mass in the pancreas or non-pancreatic tissues for glucose homeostasis.

### Example 15

### Pdx1 Treatment Promoting Endogenous β-cell Regeneration

To determine whether the regenerated islet β-cells in the pancreas play a role in the rPdx1-mediated normoglycemic mice, a group of Pdx1-mediated normoglycemic mice (n=4) were subjected to near-total (>90%) pancreatectomy around day-30 post-injection, and the removed pancreatic tissue was processed for analysis of morphology, and/or tissue insulin measurement. As shown in FIGS. 20A, blood glucose levels rose sharply following nearly total pancreatectomy, indicating that insulin-producing islet cells in the pancreas played a dominant role in attaining and maintaining normoglycemia at day-30 post rPdx1 injection. These results hinted that in vivo delivery of rPdx1 protein promoted endogenous beta-cell regeneration. Note that blood glucose rose higher in the GFP-treated mice, indicating presence of minimal spontaneous beta-cell regeneration.

Examination of histology and insulin expression on sections of paraffin-embedded pancreas tissue confirmed that vigorous islet β-cell regeneration was evident with larger and abundant islets were seen in Pdx1-treated mouse pancreata, whereas rare small islets were observed in GFP-treated mice (FIGS. 21A and 21B). Sequences of oligonucleotide primers used are shown in Table 2 (below).

TABLE 2
Primer name, sequences, size, GenBank #, and PCR condition

| | | | PCR | | | |
|---|---|---|---|---|---|---|
| | | | size | GenBank | Tm | Cycle |
| Genes | Forward primer | Reverse primer | (bp) | Acc. No. | (° C.) | No. |
| HPRT | CTCGAAGTGTTGGATACAGG | TGGCCTATAGGCTCATAGTG | 350 | NM_013556 | 56 | 40 |
| Insulin I | TAGTGACCAGCTATAATCAGAG | CAGTAGTTCTCCAGCTGGTA | 372 | NM_008386 | 56 | 40 |
| Insulin I | ATCAGAGACCATCAGCAAGCA | CTTCCTCCCAGCTCCAGTTGT | 248 | NM_008386 | 56 | 40 |
| (nest) | | | | | | |
| insulin II | GCTCTTCCTCTGGGAGTCCCAC | CAGTAGTTCTCCAGCTGGTA | 288 | NM_008357 | 56 | 40 |
| glucagon | TGAAGACCATTTACTTTGTGGCT | TGGTGGCAAGATTGTCCAGAAT | 492 | NM_008100 | 57 | 40 |
| somatostatin | CTCTGCATCGTCCTGGCTTTG | GGCTCCAGGGCATCATTCTCT | 173 | NM_009215 | 56 | 40 |
| IAPP | TGAACCACTTGAGAGCTACAC | TCACCAGAGCATTTACACATA | 282 | NM_010491 | 55 | 40 |
| Glut-2 | CGGTGGGACTTGTGCTGCTGG | GAAGACGCCAGGAATTCCAT | 412 | NM_031197 | 56 | 40 |
| P48 | CCCAGAAGGTTATCATCTGCC | CGTACAATATGCACAAAGACG | 245 | NM_018809 | 57 | 40 |
| Elastase | AATGACGGCACCGAGCAGTATGT | CCATCTCCACCAGCGCACAC | 344 | NM_033612 | 57 | 40 |
| Amylase | TGGGTGGTGAGGCAATTAAAG | TGGTCCAATCCAGTCATTCTG | 371 | NM_009669 | 56 | 40 |
| glucokinase | ATCCTGGCAGAGTTCCAGCT | CTTGTGTTTCATCTGATGCT | 373 | NM_010292 | 56 | 40 |
| Pdx1 | ACCGCGTCCAGcTCCCTTTC | CCGAGGTCACCGCACAATCT | 357 | NM_008814 | 57 | 40 |
| NeuroD1 | CATCAATGGCAACTTCTCTT | TGAAACTGACGTGCCTCTAAT | 257 | NM_010894 | 56 | 40 |
| Isl1 | AGACCACGATGTGGTGGAGAG | GAAACCACACTCGGATGACTC | 296 | NM_021459 | 56 | 40 |
| Nkx2.2 | GACAGCAGCGACAACCCCTACA | CGTGAGACGGATGAGGCTGG | 306 | NM_010919 | 56 | 40 |
| Nkx6.1 | AGAGTTCGGGTCCAGAGGT | AGTGATGCAGAGTCCGCCGT | 382 | NM_144955 | 56 | 40 |
| Pax4 | CCAGCCACAGGAATCGGACTA | TCCCTGGAGAATTTTTTGGTACT | 252 | NM_011038 | 57 | 40 |
| pax6 | GTGAATCAGCTTGGTGGTGTC | GAAGGGCACTCCCGTTTATAC | 308 | NM_013627 | 56 | 40 |
| Ngn3 | TGGCACTCAGCAAACAGCGA | AGATGCTTGAGAGCCTCCAC | 516 | NM_009719 | 56 | 40 |

Additionally, individual scattered insulin-positive cells with pancreatic exocrine cell appearance are also noted in these pancreata. Such findings indicated that, when administered in vivo in multiple doses, rPdx1 protein promotes endogenous insulin-producing β-cell regeneration via a not-yet defined molecular and cellular mechanism.

To further examine the patterns of the regenerated islets, double immunofluorescence studies were performed using anti-insulin and anti-glucagon antibodies. It is of great interest that based on the β-cell/β-cell ratio and distribution patterns, the pancreatic islets in the mice could be arbitrarily divided into three stages (FIG. 21B): Stage-1, where the pancreatic β-cell/β-cell ratio is approximately 0.2, with abundant disorganized glucagon-positive β-cells and relatively few β-cells scattered throughout; Stage-2, a β-cell/β-cell ratio ˜1, with roughly equal number of glucagon- and insulin-positive β-cells; and Stage-3, ratio of pancreatic a β-cell/β-cell ratio of 5, showing a inverse ratio, with insulin-producing β-cells predominating. The architecture of the islets from Stage-1 through Stage-3 became more organized with concurrently increased numbers of insulin-producing β-cells. The three readily discernable patterns described above appear to represent general appearance of the progression of islet cell regeneration, since these patterns were also noted in GFP-treated mice.

To determine the molecular events of rPdx1-mediated islet β-cell regeneration, the status of several genes known to be related to pancreas and β-cell regeneration as well as normal β-cell physiologic functions were assayed. Real-time PCR was used to determine their levels of expression in the pancreas. Following the treatment with rPdx1 on day-14 and day 40, the mice were sacrificed and the total RNA was extracted from the pancreata to evaluate the gene expression profile during the regeneration process (Gagliardino J. Endocrinol. 177, 249-259 (2003), Pittenger Pancreas 34, 103-111 (2007), Jonsson et al., Nature 371, 606-609 (1994), Wu et al. Mol. Cell Biol. 17, 6002-6013 (1997), Stoffers et al., Nat. Genet. 15, 106-110 (1997), Holland et al., Diabetes 54, 2586-2595 (2005)). As shown in FIG. 21C, the rPdx1 treatment of diabetic mice at day 14 resulted in markedly up-regulated levels of insulin (21.3 times), endogenous Pdx1 (3.8 times), INGAP (14.5 times), Reg3d (8.1 times), Reg3g (6.8 times), and pancreatitis-associated protein—Pap (34.3 times) relative to their corresponding control (GFP-treated pancreas) values. A similar pattern of up-regulation of the aforementioned genes was observed to be persistent at day 40 post-treatment. Interestingly, expression of Pap, although continued to be up-regulated when compared to GFP-treated mice. It was noticeably reduced at day 40 post-treatment when compared to that on day 14. Irrespective of the mechanism(s) (i.e., β-cell differentiation and replication, ductal cell neogenesis, or exocrine cell transdifferentiation) responsible for these effects, rPdx1 protein has the capacity to induce the formation of new β-cells via the up-regulation of pancreas regenerating genes and thereby alleviating the hyperglycemic condition.

### Example 16

### Pdx1 Treatment Promoting Liver Cell Transdifferentiation into Insulin-Producing Cells

Several in vivo studies have shown that virus-mediated transgene expression of Pdx1 in the liver cells result in liver cell transdifferentiation into IPCs and reverse hyperglycemia in diabetic mice (Ferber, S. et al. Nat. Med. 6, 568-572 (2000), Ber, I. et al. J. Biol. Chem. 278, 31950-31957 (2003)). However, it is unclear whether direct in vivo treatment of diabetic mice with rPdx1 protein can have similar effect to the liver cell transdifferentiation. To determine the effect on the liver of administered rPdx1 at day-14 post-injection, liver tissues were obtained from mice treated with either Pdx1 or GFP and these samples were examined for the presence of IPCs immunohistochemically with anti-insulin antibody. Multiple intraperitoneal injections of soluble Pdx1 protein reversed hyperglycemia to nearly normal glycemia. FIG. 22A shows representative liver micrographs from mice treated with PTD-GFP (left column) or Pdx1 (right two columns). Most scattered insulin-staining positive liver cells were distributed along the edges of central veins (marked C.V. in micrographs), a pattern consistent with rPdx1 tissue distribution in the liver (see FIG. 22B). There were scattered individual insulin-positive hepatocytes (Black arrows) with small bi-nuclei and condensed chromatin, hinting more mature cell pattern. These cytologic features were in sharp contrast to those adjacent insulin-negative and active hepatocytes with larger nuclei and a more open chromatin pattern (arrows). No insulin-producing cells were observed in the control GFP-treated mouse liver.

Next the expression profile of pancreatic genes in the Pdx1 or GFP-treated livers was investigated by comparing the results at day-14 and day-40 post-protein injection (FIG. 22B). The Pdx1-treated livers at day-14 exclusively expressed many pancreatic genes, including endogenous Pdx1, insulin I, glucagon, elastase, and IAPP, as well as up-regulated expression of other pancreatic endocrine genes (insulin II, somatostatin, NeuroD, and Isl-1) and pancreatic exocrine genes (p48 and amylase), as compared to the gene expression of GFP-treated livers. Ngn3 gene expression was not detectable. Interestingly, by day-40, the expression of insulin I, glucagon, elastase, and IAPP genes was not observed, while the other aforementioned pancreatic gene expression continue to be present, albeit at reduced levels. These data suggested that the insulin produced by rPdx1-mediated liver cells in the early stage may play an important role for reducing hyperglycemia and promote pancreas β-cell regeneration.

### Example 17

### Effects of rPdx1 Protein on Other Vital Organs

The findings presented above demonstrated that, when delivered in vivo, rPdx1 has a genuine therapeutic effect on diabetic mice by promoting endogenous islet cell regeneration and liver cell transdifferentiation into IPCs. Given the intrinsic ability of rPdx1 protein to penetrate cells indiscriminately, the specificity of rPdx1 effect on various organs was also examined for expression of pancreatic genes, including Pdx1, insulin I, glucagon, and amylase. After harvesting tissues from heart, brain, kidney, lung, gut, and spleen from the mice receiving rPdx1 at day-14 post-treatment, the total RNA for gene expression studies by RT-PCR was extracted. The rationale behind this approach was that, if intraperitoneal rPdx1 treatment were to result in indiscriminate activation of pancreatic key genes, such findings would cast doubt on the significance of the aforementioned observations of pancreatic genes in the liver (FIG. 22B). As shown in FIG. 22C, little or no indication of pancreatic gene activation by rPdx1 treatment in heart, brain, kidney, lung, gut, and spleen was found. These results suggest that Pdx1-treatment protocol was efficacious with respect to restoration of normoglycemia through promoting the liver cell transdifferentiation and β-cell regeneration, without any detectable evidence of undesired systemic toxicity.

### Example 18

### Complementary Relationship between Pancreas and Liver at Tissue Insulin Levels

To determine the relative contribution of pancreas and liver tissue-derived insulin to ameliorating blood glucose levels after rPdx1 treatment, pancreata and livers from the Stz-treated mice sacrificed around Day-14 or Day-40 after Pdx1 injection were extracted in acidic ethanol, and the resulting insulin content was measured with an ultra-sensitive ELISA kit for mouse insulin. FIG. 23A shows that pancreatic insulin content in Pdx1-treated diabetic mice at day-14 and at day-40 post-injection was approximately 44% and 68%, respectively, of the normal pancreas levels, amounting to 6.7 times higher level at day-14 and 15.8 times higher at day-40 post-treatment, as compared to the GFP-treated control mice (12.0 ng/mg and 9.5 ng/mg). These findings indicate that in vivo rPdx1 treatment promoted islet β-cell regeneration in the pancreas. This increased production of pancreas insulin is statistically significant when compared to GFP-treated mice in both day-14 and day-40 post-treatment. These results were also consistent with our finding of rebound hyperglycemia following near-total pancreatectomy (FIG. 21A).

With diabetic mice treated similar to the pancreas insulin content measurements, liver tissue insulin contents at day-14 and day-40 post-injection were examined. As shown in FIG. 23B, indeed, there is marked increase (˜16 folds) in the liver tissue insulin content at day-14 post-treatment in the Pdx1-treated mice over the GFP-treated mice and nearly nine-fold increase than normal liver. Interestingly, there was sharply reduced liver insulin content at day-40 post-Pdx1-treatment, although it was still 7.3-times higher than GFP-treated mice and approximately two times higher than normal liver. This increase in liver insulin production is statistically significant when compared to insulin levels in normal or GFP-treated mice.

The generation, purification, and characterization of rPdx1 protein is shown in FIGS. 24A-24C. Pdx1 tissue distribution is shown in FIGS. 25A and 25B.

### Example 19

### Pdx1 Treatment Does Not Induce Pancreatic Gene Expression in Healthy Mice

Normal mice were injected with large dose of rPdx1 protein in two ways. First, mice were injected with single large dose (10 mg) of purified rPdx1 protein and 2^{nd}, mice were injected with 1 mg/day for 10 consecutive days. The mice were observed for body weight, well being, and other indicators including blood glucose levels, liver and myocardial enzymes to monitor the potential toxicity. There were no noticeable changes in the above parameters when compared with normal mice that were not injected with Pdx1 protein. All mice were sacrificed at day 14 post-injection and the key pancreatic gene expression was examined in the livers. Because at Day-14, the livers from mice injected with rPdx1 strongly expressed pancreatic genes, including insulin, glucagon, amylase, and endogenous Pdx1. To determine whether these key pancreatic genes were expressed in the normoglycemic mice receiving large dose of rPdx1 injection, the total RNA from livers of both group of mice was harvested and gene expression was determined by RT-PCR as shown in FIG. 26. No effect on blood glucose levels was observed in control mice receiving PTD-GFP injections. There was no detectable expression of the examined pancreatic genes in the livers. This indicated that there was no toxicity associated with the in vivo delivery of rPdx1 protein to normal individual mice. This result suggests that rPdx1 protein therapy works only in diabetic animal and has no effect on normal mice.

The experiments described above are carried out using the following materials and methods.

### Intraperitoneal (i.p.), and Portal Vein (p.v.) Delivery of PTD-Fusion Proteins

Mice were induced to be hyperglycemic (>350 mg/ML) by daily intraperitoneal injection of streptozotocin (Stz) for five days at 50 μg/g of body weight. Normal or diabetic mice were then anesthetized and intraperitoneally injected with the indicated amounts (50-200 μg/mouse) of PTD-GFP or PTD-PTF fusion protein in 500 μl in PBS. See (Kay et al., Hum Gene Ther 1992; 3(6):641-647). Blood glucose levels were monitored via tail vein tapping. Various tissues, including pancreas and liver, were subsequently collected for immunohistochemical analysis for the presence of insulin and glucagon-producing cells.

For portal vein delivery of PTD-PTF fusion proteins, five to six-week-old mice are anesthetized with isoflurane, and surgical procedures are performed according to Institutional Animal Care and Use Committee protocols. The animals are shaved and a 1-2 cm incision is made in the upper abdominal area to expose the portal vein. A special catheter is placed into one of the main branches in the portal vein and the outlet is linked to an Alzet Osmotic pump (Theeuwes et al., Ann Biomed Eng 1976; 4(4):343-353) which is embedded under the skin of the mouse belly. PTD-fusion proteins are delivered into the portal vein via the catheter in a constant rate for various times. For diabetic mice, blood glucose levels are monitored and IPGTT is performed after the blood glucose is normalized as previously described in Cao et al., Diabetes 2004; 53(12):3168-3178 and Tang et al., Lab Invest 2006; 86:83-93. At certain time points, mice are sacrificed for collecting tissues of the liver, spleen, kidney, duodenum, and pancreas, as well as implanted reprogrammed hepatic cells. The tissues will be used for analysis of morphology, immunostaining (paraffin sections), and gene expression and for determination of tissue pancreatic hormone content (frozen tissue) such as insulin, glucagon and amylase by ELISA.

### Implantation of Osmotic Mini-Pumps and PTD-PTF Delivery

Chronic administration of PTD-PTF fusion proteins is accomplished by subcutaneous implantation, under isoflurane anesthesia, of an Alzet osmotic mini-pump (Alza, Model no. 2001-1 week, or 2002-2 weeks) (Theeuwes et al., Ann Biomed Eng 1976; 4(4):343-353; Heinrichs et al., Proc Natl Acad Sci USA 1996; 93(26):15475-15480) with the outlet of the infusion pump attached to a catheter to allow the PTD-PTF fusion proteins to be infused continuously into the portal vein or intraperitoneal cavity. The Alzet osmotic pump provides around-the-clock exposure to test agents at predictable levels; permits continuous administration of short half-life proteins, convenient method for chronic dosing of laboratory animals; minimizes unwanted experimental variables and ensure reproducible, consistent results; eliminates the need for nighttime or weekend dosing; reduces handling and stress to laboratory animals; is small enough for use in mice or very young rats; and allows for the targeted delivery of agents to virtually any tissue. The pump is implanted sub-dermally to infuse PTD-PTF fusion protein (dissolved in PBS or with PBS alone as the control vehicle) at a rate of 1 ul/hour for 1 week or 0.5 ul/hour for 2 weeks. To achieve a PTD-PTF dose of 10 ug/g body weight/day, a stock solution of 5-10 mg/ml is used for a mouse with a body weight of 20 g and adjusted accordingly. The osmotic pump is activated before implantation by overnight incubation in a 37° C. water bath to standardize the initiation and rate of PTD-PTF infusion over time.

### Western Blot

The PTD-PTF pump is implanted at 1600 hours, and blood glucose levels are examined 24 hours following pump removal. Mouse tissues are collected and proteins are extracted with PBS homogenization buffer (1% Triton-X, 1 mM phenylmethylsulfonyl fluoride, and protease inhibitor cocktail). The tissue lysates are centrifuged at 20,000 rpm for 30 minutes at 4° C. The protein concentration of the supernatant is determined in a standard protein assay carried out using a Bio-Rad protein reagent. The supernatant is mixed with an equal volume of 2× SDS sample buffer and heated to 95° C. for 5 minutes. Heated mixtures are then centrifuged at 15,000 rpm for 5 minutes to remove insoluble materials. The supernatants are analyzed using SDS-polyacrylamide gel electrophoresis. The proteins are transferred to a nitrocellulose membrane and blotted with anti-V5 antibody (1:5000) or with anti-GFP antibody then visualized using electrochemiluminescence, Amersham ECL luminescence system (Amersham Pharmacia Biotech, Buckinghamshire, UK). Fusion protein levels are quantified by densitometry using a Densitometer Scanning Instrument.

### RT-PCR, Histology, Immunohistochemistry and Fluorescence Microscopy.

These methods are well established in our laboratory and details can be found in the following publications, which are incorporated by reference: Cao et al., Diabetes 2004; 53(12):3168-3178; Tang et al., Lab Invest 2006; 86:83-93. For visualization of GFP protein in tissue sections, the mouse tissue was fixed overnight in formalin and transferred into 50% sucrose 12 hours. The tissue was cut as frozen sections and the slides were recovered with DAPI-containing mounting medium. The GFP-containing cells will be observed and photographed under the fluorescence microscope.

### Plasmids and Plasmid Construction

Pdx1-VP16 was constructed by fusing the activation domain of VP16 (80 amino acids) to the mouse COOH-terminus of Pdx1 as follows. Full-length Pdx1 was isolated from IPF1-pcDNA3 using the T7 primer and a 3′ primer that included a ClaI site, 5′-TCG CAG TGG ATC GAT GCT GGA G-3′. The product was cut with HindIII and ClaI and subcloned into VP16-N in pCS2⁺ (Diabetes. 2004; 53: 3033-3345). Pdx1-VP16 was then subcloned into the HindIII and XbaI sites of pcDNA3. The PTD sequence was appended to this sequence.

### Adeno-Associated Virus Serotype 2 (AAV2) Vectors.

All AAV2-PTF plasmids containing mouse Pdx1, Pdx1-VP16, Ngn3 coding sequence were constructed in our laboratory. The quality and titer of these viruses have been confirmed. In brief, the coding sequences of GFP and PTFs (mouse Pdx1, mPdx1-VP16, and mNgn3), beginning with a Xba I site and ending with a Hpa I site, were amplified from their corresponding pCRT-cDNA plasmids by pfu using suitable pairs of forward and reverse primers. After add-A reaction, these PCR products were ligated with pCR2.1-TOPO vector.

Positive colonies containing correct inserts and AAV backbone plasmid (pUF11) were digested by Xba I and Hpa I. Both 6.2 kb pUF11 vector and the DNA inserts (mPdx1, mPdx1-VP16, and mNGN3) were recovered by gel purification and then were ligated according to standard procedure. The coding sequences and correct direction from selected positive colonies were confirmed by restricted enzyme digestion and sequence analysts. The AAV-PTF vector plasmids have a chicken β-actin promoter with a CMV enhancer. AAV2 vectors were generated by triple plasmid transfection of 293 cells. The AAV2-PTF viruses were purified with CsCl gradient ultracentrifugation and the viral particle (VP) titer was determined as genomic copies per ml by real-time PCR. The purity of vectors was assessed by silver-stained SDS-PAGE. The concentrated vectors were aliquoted, and stored at −80° C. for in vivo studies.

Construction and Production of rPdx1 Protein.

The full-length the mouse PDX1 cDNA was amplified by PCR and subcloned into the Nde I and Xho I sites of pET28b (Novagen). BL21 (DE3) cells containing the expression plasmids were grown at 37° C. to an optical density O.D₆₀₀ of 0.8. Isopropyl β-D-1-thiogalactopyranoside (IPTG) was added to a final concentration of 0.5 mmol/L, and the cells were then incubated at 18° C. for 18 hours. Bacteria were lysed by pulse sonication in Buffer A containing 5 mM Imidozole and proteinase inhibitors (Roche Diagnostics). The supernatant of bacterial lysates obtained after centrifugation were applied to a column of Ni-nitrilotriacetic acid (Ni-NTA) agarose (Invitrogen) and washed with several volumes of wash buffer (buffer A containing 25 mM imidazole). The protein was eluted by elution buffer containing 250 mM of imidazole. The purity of the eluted protein fractions were characterized by SDS-PAGE/Coomassie Blue staining after dialysis against PBS.

### Construction and Production of PTD-GFP Protein.

The PTD-GFP plasmid with 11 amino acids (YGRKKRRQRRR) of HIV-1 TAT at the N-terminus of GFP was constructed by the PCR method. The PCR fragment was directly cloned into a pT7/CT-TOPO expression plasmid (Invitrogen). Protein production and purification was carried out largely as described for rPdx1 protein.

Preparation of pNeuroD-GFP and Mouse Pdx1 Lentiviral Vectors.

Lentiviral vector (LV) containing the cDNA coding sequence of mouse PDX1 was constructed as previously described (Tang et al., Lab Invest 86, 83-93 (2006)). A 950 bp reporter construct (−940 to +10) of human NeuroD/Beta2 promoter (Miyachi et al., Brain Res. Mol. Brain Res. 69, 223-231 (1999)) was cloned by PCR and ligated to GFP. LV was constructed by inserting the pNeuroD-GFP gene into a pTYF vector cassette. Lentivirus was produced and the titer determined as previously described (Tang et al., Lab Invest 86, 83-93 (2006)).

Cell Entry and Immunoblotting.

WB cells (rat liver epithelial stem cells) at 70% confluence were treated with purified rPdx1 (0.2 μm) for 15-minutes, 30-minutes, 1-hour, 2-hours, 6-hours, 12-hours, and 24-hours. The cells were washed three times with PBS and harvested in cell lysis buffer (150 mM NaCl, 50 mM Tris-HCl, pH 7.5, 500 μM EDTA, 1.0% Triton X-100, and 1% sodium deoxycholate) containing a protease inhibitor mixture (Roche Diagnostics). The rPdx1 in cell lysates (50 μg/lane), for cell entry studies, was detected by Western blotting as previously described^{1,3} with rabbit anti-Pdx1 serum (1:1000, raised with purified mouse rPdx1) or anti-His antibody (1:2000, Invitrogen).

Cell Transduction and Flow Cytometric Analysis.

WB cells at 70% confluence were first transduced by LV-pNeuroD-GFP at a multiplicity of infection of 20 following the procedure as previously described¹. The transduced WB cells then were incubated in the presence or absence of 0.5 μm rPdx1 protein. The pNeuroD-GFP containing WB cells were transduced with LV-Pdx1, which served as positive control for cell-made Pdx1 protein. The cells were harvested at 72-hrs post-treatment, fixed in 1% formaldehyde for 10-minutes, followed by washing 3× with PBS plus 1% BSA, and resuspended for flow cytometric analysis. Cytospin slides were prepared with the treated cells and covered with mounting medium containing DAPI.

Animal Studies.

Balb/c mice (8-10-wk-old) were injected with streptozotocin (Stz) (Sigma) at a dose of 50 mg/kg body-weight (bw) for 5 consecutive days to induce diabetes as previously described^{1,3}. The animals with fasting blood glucose levels around 300 mg/dL for two consecutive readings received protein treatment. The diabetic mice were i.p. injected with either purified rPdx1 or GFP protein (0.1 mg/day/mouse) for 10 consecutive days. The fasting blood glucose levels were measured regularly using a glucometer after the mice were deprived from food for 8-hrs. The sequential experimental events of the animal studies are summarized in Scheme 1 (FIG. 25C).

Intraperitoneal Glucose Tolerance Test (IPGTT).

The mice were fasted for 8-hrs before the IPGTT was performed. The normal, rPdx1-, or GFP-treated mice were i.p. injected with glucose (1 mg/g bw) and the blood glucose levels were measured at 5, 15, 30, 60, and 120-minutes post-injection. Subtotal pancreatectomy (˜90%) was performed under generalized anesthetic conditions. The mice were scarified at various time-points, and organ tissues and blood were collected for later studies of histology, gene expression, pancreas/liver tissue contents, and serum insulin levels.

Pdx1 Protein In Vivo Kinetics and Tissue Distribution.

Mice were i.p. injected with 0.1 mg or 1 mg of rPdx1. Blood samples were drawn at 15-minutes, 30-minutes, 1-hour, 2-hours, 6-hours and 24-hours. The organ tissue sections from normal and rPdx1-treated mice were harvested, fixed in 10% formalin, and embedded in paraffin for Pdx1 immunostaining with anti-Pdx1 antibody (1:1000). The serum was separated from blood samples, 30 μl/lane was loaded in SDS-PAGE gels to separate serum proteins, and then Western blotting with anti-Pdx1 antibody was done as described above.

RT-PCR.

Total RNA was prepared from mouse tissues of the pancreas and liver using Trizol reagent, and cDNA was synthesized using superscript III reverse transcriptase (Invitrogen, Calif.) with random hexamer primers. Liver gene expression was determined by RT-PCR as previously described (Cao et al., Diabetes 53, 3168-3178 (2004)). The forward and reverse PCR primers were designed to be located in different exon(s), and their sequences are listed in supplemental data (Table 1). No RT, positive, and blank controls were included in all RT-PCR assays. Results represent at least three independent experiments.

Quantitative Real-Time RT-PCR Analysis.

cDNA from pancreatic tissues of GFP-treated control and rPdx1-treated mice was subjected to three independent PCR reactions, in duplicate or triplicate, in a thermocycler sequence detection system (MJ research Inc. DNA engine opticon 2) using SYBR green as an analyzer for the amount of PCR product during each cycle. The primers were designed in accordance to the real time PCR conditions, and the sequences are listed in (Table 1). For the real time PCR, an initial denaturation temperature of 95° C. for 15-minutes was required to activate the enzyme and an annealing temperature of 56° C. was used for all the primer pairs. The PCR amplification was carried out for 38 cycles.

Immunohistochemistry and Immunofluorescence.

The tissues from various organs were fixed in 10% formalin for 24-hours and transferred to PBS before paraffin blocks were made. Sections (5 μm) were incubated with anti-swine insulin (1:1000, Dako), anti-Pdx1 (1:5000, gift of C. V. Wright), anti-glucagon (1:200, Dako) primary antibodies, and followed by incubation with anti-mouse or rabbit IgG (1:5,000) secondary antibodies conjugated with horse radish peroxidase HRP (Dako). The specific immunostaining was visualized using DAB substrate kit (Dako) as previously described³. For double immunofluorescence, the paraffin embedded sections (5 μm) were incubated with guinea pig anti-swine insulin (1:200, Dako) and goat anti-glucagon (1:50, Santa Cruz) primary antibodies overnight at 4° C., and followed by donkey anti-guinea pig IgG conjugated with FITC (1:1000, RDI) and donkey anti-goat with alexa fluor 594 flourochrome secondary antibodies (1:500, Invitrogen, Molecular probes).

### Tissue and Serum Insulin Measurements by ELISA.

Whole organs of pancreas and liver were harvest, weighed, and placed immediately in acid-ethanol solution (180 mM HCl in 70% ethanol) on ice with corresponding volume (1 ml buffer/0.1 g liver or 0.05 g pancreas) according to published procedure with minor modifications⁴. Tissue insulin levels were measured using an ultrasensitive mouse insulin ELISA kit (ALPCO) according to the manufacture's instructions. The optical absorbance was measured immediately by a BIO-RAD 3550-UV microplate reader. The final results were converted to ng insulin/mg pancreas tissue or ng insulin/gram liver tissue.

For measurement of serum insulin, both normal and treated mice were first starved for 6-hr, and blood samples were collected at 15-min following a glucose (1 mg/g bw) stimulation. The levels of serum insulin were determined as described in tissue insulin ELISA.

### Statistical Analysis.

The statistical significance of our experimental findings was analyzed by using an independent sample t-test, requiring a P value of less than 0.05 for the data to be considered statistically significant.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. In particular, the entire disclosures of Cao et al., Diabetes 2004; 53(12):3168-3178; Tang et al., Lab Invest 2006; 86:83-93; and International Patent Publication No. WO 2005/083059 is incorporated herein by reference in their entirety.

1.-90. (canceled)

91. A method for reprogramming a cell, the method comprising:
(a) contacting the cell with a transcription factor fusion protein comprising a homeodomain or fragment thereof fused to an antennapedia protein transduction domain, wherein the transcription factor fusion protein has at least 85% amino acid identity to a Pdx-1 polypeptide and has transcriptional regulatory activity; and
(b) altering the expression level of at least one polypeptide in the cell, thereby reprogramming the cell.

92. The method of claim 91, wherein the cell is selected from the group consisting of adipocytes, bone marrow derived cells, epidermal cells, endothelial cells, vascular cells, fibroblasts, hematopoietic cells, hepatocytes, gut cells, myocardial cells, myocytes, neurons, pancreatic cells, spleen cells, and their progenitor cells or stem cells.

93. The method of claim 91, wherein the cell is contacted in vitro or in vivo.

94. The method of claim 91, wherein the alteration is an increase in the level of a polypeptide that is not detectably expressed in a corresponding control cell.

95. The method of claim 91, wherein the reprogrammed cell expresses insulin.

96. The method of claim 91, wherein the cell is a tissue cell or organ cell.

97. The method of claim 96, wherein the organ is spleen, heart, lung, liver, kidney, brain, pancreas, vascular system, or a progenitor or stem cell thereof.

98. The method of claim 96, wherein the organ is liver or pancreas.

99. The method of claim 91, wherein the reprogramming is by the transdifferentiation of a liver or pancreas cell.

100. The method of claim 91, wherein the transcription factor fusion protein has at least 90% amino acid identity to a Pdx-1 polypeptide.

101. The method of claim 91, wherein the transcription factor fusion protein has at least 95% amino acid identity to a Pdx-1 polypeptide.

102. The method of claim 91, wherein the transcription factor fusion protein is at least 90% identical to a human Pdx-1 polypeptide.

103. A method for generating an insulin producing cell in a mammal for the treatment of hyperglycemia, the method comprising:
(a) contacting an organ or tissue with a pancreatic transcription factor or fragment thereof comprising a protein transduction domain; and
(b) increasing the expression of insulin in a cell of the organ or tissue, thereby generating an insulin producing cell.

104. The method of claim 103, wherein the transcription factor is selected from the group consisting of Pdx-1, Pdx-1/VP16, Ngn3, Pax4, NeuroD1, Nkx2.2, Nkx6.1, IsI1, Pax6, MafA, and NGN3.

105. The method of claim 103, wherein a cell of the organ or tissue show an increase in the expression of one or more genes selected from the group consisting of Pdx1, INGAP, Reg3d, Reg3g, Pap, insulin I, glucagon, elastase, IAPP, insulin II, somatostatin, NeuroD, IsI-I pancreatic exocrine genes, p48 and amylase.

106. The method of claim 103, wherein the organ is liver or pancreas.

107. The method of claim 103, wherein the insulin producing cells are generated by the transdifferentiation of a liver or pancreas cell.

108. A method of inducing cell regeneration in a subject in need thereof, the method comprising administering an effective amount of Pdx1 protein to the subject and inducing the regeneration of a cell.

109. The method of claim 108, wherein PDX1 administration increases the expression of a gene selected from the group consisting of: Pdx1, INGAP, Reg3d, Reg3g, pancreatitis-associated protein—Pap, insulin I, glucagon, elastase, IAPP, insulin II, somatostatin, NeuroD, Isl-1 and pancreatic exocrine genes p48 and amylase.

110. The method of claim 108, wherein the method increases pancreatic cell function.

111. The method of claim 108, wherein the cell is a β-cell, pancreatic cell, or liver cell.

112. The method of claim 108, wherein the administration results in normoglycemia.

113. A method of inducing liver cell regeneration in a subject in need thereof, the method comprising increasing the expression of one or more genes selected from the group consisting of: Pdx1, INGAP, Reg3d, Reg3g, pancreatitis-associated protein—Pap, insulin I, glucagon, elastase, IAPP, insulin II, somatostatin, NeuroD, Isl-1 and pancreatic exocrine genes p48 and amylase, thereby inducing liver cell regeneration.

114. The method of claim 113, wherein the method increases liver cell function.

115. A method for reprogramming a cell, tissue, or organ, the method comprising:
contacting the cell, tissue or organ with
(a) an effective amount of a transcription factor polypeptide comprising a DNA-binding domain, a homeodomain, transactivation domain or an antennapedia domain; and
(b) an endogenous or a recombinant pancreatic transcription factor comprising a basic helix-loop-helix (bHLH) domain or a homeodomain.

116. The method of claim 115, where the transcription factor polypeptide of (a) is a Pancreatic Homeodomain Containing Protein-1 (Pdx-1), PDX-1/VP16, or a biologically active fragment thereof comprising at least a homeodomain and an antennapedia domain.

117. The method of claim 115, wherein the transcription factor of (b) is Neurogenin 3 (Ngn³).

118. The method of claim 115, where the tissue or organ is selected from the group consisting of liver, pancreas, gut, and lung.

119. A method for reprogramming a cell, the method comprising:
(a) contacting the cell with a transcription factor fusion protein having at least 85%, 90%, or 95% amino acid identity to murine Pdx-1 polypeptide GenBank Accession No. NP_{—}032840; and
(b) altering the expression level of at least one polypeptide in the cell, thereby reprogramming the cell.
